(19) United States
(12) Patent Application Publication
(10) Pub. No.: US 2013/0018463 A1
(43) Pub. Date: Jan. 17, 2013

(54) COLLAGEN FIBER CONSTRUCTS FOR REPLACING CRUCIATE LIGAMENTS

(71) Applicants: Daniel Roland Haddad, Wuerzburg (DE); Meike Haddad-Weber, Wuerzburg (DE); Ulrich Noeth, Wuerzburg (DE)

(72) Inventors: Daniel Roland Haddad, Wuerzburg (DE); Meike Haddad-Weber, Wuerzburg (DE); Ulrich Noeth, Wuerzburg (DE)

(21) Appl. No.: 13/517,248

(22) PCT Filed: Dec. 20, 2010
(86) PCT No.: PCT/EP2010/070312

§ 371 Date: Sep. 26, 2012

(30) Foreign Application Priority Data
Dec. 21, 2009 (DE) 10 2009 059 901.0

(51) Int. Cl. A61F 2/08; C07K 14/78; D02G 3/38; D02G 3/44; D02G 3/02; D02G 3/26

(52) U.S. Cl. 623/13.12; 87/12; 442/304; 428/365; 87/8; 19/144; 623/13.2; 530/356

(57) ABSTRACT

The present invention relates to a collagen fiber construct composed of single collagen fibers, which is sterilized with alcohol and via irradiation and not populated with cells, wherein the single collagen fibers are isolated from collagen-containing tissue from mammals. The present invention also relates to a method for manufacturing a collagen fiber construct composed of single collagen fibers, which is sterilized with alcohol and via irradiation and is not populated with cells, wherein the single collagen fibers are isolated from collagen-containing tissue from rat tails. Finally, there is also described the use of the collagen fiber constructs as xenoimplants.

The present invention relates to a collagen fiber construct composed of single collagen fibers, which is sterilized with alcohol and via irradiation and not populated with cells, wherein the single collagen fibers are isolated from collagen-containing tissue from mammals. Furthermore, the present invention relates to a collagen fiber construct wherein the single collagen fibers are isolated from rat tails. Moreover, there are comprised collagen fiber constructs wherein several single collagen fibers are knotted into a collagen thread. Furthermore, the present invention comprises a collagen fiber construct wherein one or several collagen threads are knitted into a collagen cord, which threads can in turn be twisted into a collagen cord. The present invention also relates to a method for manufacturing a collagen fiber construct composed of single collagen fibers, which is sterilized with alcohol and via irradiation and is not populated with cells, wherein the single collagen fibers are isolated from collagen-containing tissue from rat tails. Finally, there is also described the use of the collagen fiber constructs as xenoimplants. In particular, the present invention relates to collagen fiber constructs wherein said constructs are preferably cruciate ligament constructs.

Every year there are 60,000 cruciate ligament ruptures in Germany, more than 200,000 in the USA and 75,000 in Japan. The anterior cruciate ligament (ACL) is one of the essential stabilizing structures of the knee joint. Hence, an ACL injury leads to instability of the joint, which leads to damage to the secondary stabilizers (in particular the internal meniscus) and, finally, to gonarthrosis (Woo, et al., Clin Orthop Relat Res, p312-323 (1999)). The possibilities for spontaneous healing of the ligament after a rupture are limited. Hence, manifold approaches have been pursued for replacing the injured cruciate ligament by other structures. From the mid eighties on, allogeneic tendon implantations (often implants obtained from cadavers) were carried out. In an allogeneic implantation the implanted tissue does not stem from the recipient himself, but from a donor of the same species. An essential problem of allogeneic implantation consists in the transmission of pathogens and in a possible rejection reaction due to a lack of correspondence between the features recognized by the immune system and the recipient's tissue. Because of the high risk of virus infections, allo-implants are primarily used today only in the USA (Laurencin, et al., Biomaterials 26, 7530-7536 (2005)). Furthermore, allogeneic implants have a reduced tear strength due to the sterilization methods and storage (cryopreservation=storage of implants at down to −135° C.) (Barbour and King, Am. J. Sports Med. 31, 791-796 (2003)). Likewise, diverse experiments have been done with synthetic ligament materials such as silk or Problast as a sole replacement and as an augmentation of tendon grafts. However, these showed poorer long-term results compared to autologous tendon grafts (grafts of one's own tissues). Up to this day, autologous ligament replacement with bone-tendon-bone-patellar tendon grafts and semitendinosus (hamstring) and gracilis tendon grafts has become accepted as the best possible treatment of cruciate ligament ruptures at present and is the surgical standard (Woo et al., J Orthop Surg 1, 2 (2006)). An essential problem of both techniques is the donor site morbidity, because the additional operative procedure for tissue removal is frequently associated with healing problems. This donor site morbidity is found in particular with patellar tendonoplasty (Laurencin et al., Biomaterials 26, 7530-7536 (2005) and Butler et al., J Orthop Res, 26, 1-9 (2007)). The cruciate ligament construct of braided structure and composed of PLAA (poly-L-lactic acid) as described by Laurencin, et al., Biomaterials 26, 7530-7536 (2005) was not tested in vivo. Furthermore, autologous tendon grafts are subject to intra-articular remodeling, which leads to a change in the tendon structure and to a reduced mechanical load capacity (Roseti et al., J Biomed Mater Res A 84, 117-127 (2008)). Permanent replacement by synthetic ligament prostheses has not proved successful in particular due to a synovitis induced by material abrasion, and material failure.

Braided or twisted collagen fiber constructs which consist of single collagen fibers using collagen fibers treated with so-called cross-linkers are described in Chvapil et al., Journal of Biomedical Materials Research 27, 313-25 (1993) (referred to hereinafter as (Chvapil et al. (1993)). The construct is sterilized with ethylene oxide. It is described that these cross-linkers are to increase the mechanical stability (inter alia, tear strength) of the collagen fibers or the constructs made from the fibers. However, it was simultaneously observed that collagen constructs composed of collagen fibers that have been strongly purified and strongly cross-linked are incorporated more poorly than constructs composed of collagen fibers that have been less strongly purified and less strongly cross-linked. Moreover, in the constructs described therein there was observed a clear reduction in the tear strength of these constructs after implantation. It is also described that more than one third of the constructs had wholly or partly torn after the in vivo phase. In the remaining, still intact constructs the tear strength after the in vivo phase was on average only 102 N, i.e. approx. 10% of the initial tear strength. The maximum value achieved in an animal six months after implantation was 210 N, i.e. 21% of the initial tear strength. On the basis of the results Chvapil et al. (1993) concludes that a cruciate ligament replacement composed of pure collagen fibers is unrealizable due to the fast loss or decline of mechanical tear strength. Chvapil et al. (1993) therefore proposes a composite material composed of collagen fibers and synthetic fibers.

Further, there is described in WO 2010/009511 A1 a woven collagen construct which is areally interwoven or knitted, sterilized with alcohol and which withstands a maximum tensile load (tensile load strength) of 140 N. The construct described therein has an “areal” character and serves to cover relatively large areas (e.g. for wound healing). For use as a cruciate ligament replacement the stated maximum tensile load is grossly insufficient. The in vivo application thereof was not tested.

Gentleman et al., Biomaterials, 24, 3805-13 (2003)) describe collagen fibers and collagen constructs composed of bovine Achilles tendon collagen fibers or of rat tail collagen fibers, whereby several collagen fibers are arranged parallel and knotted at the ends. The constructs were not tested in vivo and not implanted in a living organism.

It is therefore the object of the present invention to provide means and methods for manufacturing, obtaining and isolating graft materials as an alternative to autologous grafts.

This technical object is achieved by the provision of a collagen fiber construct composed of single collagen fibers, which is sterilized with alcohol and/or via irradiation and not populated with cells, wherein the single collagen fibers are isolated from collagen-containing tissue from mammals. In a preferred embodiment, the present invention thus relates to a collagen fiber construct composed of single collagen fibers, which is sterilized with alcohol and via irradiation and not populated with cells, wherein the single collagen fibers are isolated from collagen-containing tissue from rat tails.

Therefore, the core of the invention is the manufacture of a collagen fiber construct, preferably of a cruciate ligament construct, and, in a further preferred embodiment, of an anterior cruciate ligament construct, from collagen fibers from mammals. Advantageously, these cell-free constructs are pathogen-free and immunogen-free. The advantage of such constructs over previous autologous treatment methods therefore lies primarily in the lack of donor site morbidity. Moreover, an advantage of the constructs over allogeneic implants lies in the lack of risk of rejection reactions and of the transmission of infectious diseases.

As illustrated in the examples, it is surprisingly shown in in vivo grafting experiments that in particular the herein-described collagen fiber constructs have a number of advantages over the constructs described in the prior art. In particular one collagen fiber construct shows these advantages. As described more precisely hereinafter, this construct is manufactured from collagen fibers which were connected by knotting into a collagen thread (referred to hereinafter as “cruciate ligament type 1”) and subsequently knitted into a collagen cord, whereby the cords were subsequently coiled several times and finally twisted (referred to hereinafter as “cruciate ligament type 2”). It is surprisingly shown here that all animals with in particular the above-described collagen fiber construct have an intact “cruciate ligament replacement” and that there are no inflammatory reactions. Moreover, the tear strength of the collagen constructs surprisingly lay in the range of the initial tear strength of the constructs prior to implantation, or could even be increased. Thus, it was surprisingly shown that the herein-described constructs, unlike those described in the prior art, are characterized by a constant tear strength and a very good incorporation potential. Furthermore, the constructs used, as surprisingly shown in the examples, are accepted well by the bodies of the laboratory animals and a ligamentization can be observed.

The solution to the technical problem by the herein-described collagen fiber constructs, in particular by those that were knitted into a collagen cord, is also surprising insofar as Chvapil et al. (1993) considers a cruciate ligament replacement composed of pure collagen fibers to be unrealizable. However, the herein-described constructs show that there can in fact be realized a cruciate ligament replacement composed of pure collagen fibers, without the use of synthetic fibers, which moreover has the above-described advantageous and surprising properties.

The term “collagen-containing tissue” comprises here not only the tissue of mammals and, in a preferred embodiment, that from rat tails. The term also relates to tissue from other organisms and body parts. Thus, the collagen-containing tissue can stem preferably from kangaroos, bovine animals and humans. In a preferred embodiment, the collagen-containing tissue is isolated from rat tails.

The term “not populated with cells” comprises here not only collagen fibers that are completely cell-free or bear no cells at all. The term also comprises collagen fibers that bear relatively small, minimal amounts of cells. This minimal amount is preferably up to no more than 1% of the total collagen mass. In a strongly preferred embodiment, the minimal amount is up to no more than 0.3% of the total collagen mass.

In conformity with the foregoing and as illustrated further in the examples, the isolation and sterilization of the single collagen fibers and the manufacture of the collagen fiber constructs optionally comprises the following steps: (a) isolating collagen-containing tissue; (b) extracting individual and/or several single collagen fibers from the collagen-containing tissue; (c) incubating the single collagen fibers in an isotonic or iso-osmolar solution, whereby, in a further special embodiment, the incubation of the collagen fibers is effected in a 0.9% NaCl solution or phosphate buffered saline (PBS), whereby this isotonic or iso-osmolar solution is preferably sterilized; (d) sterilizing the single collagen fibers in alcohol; (e) optionally repeating the washing and sterilization steps according to points (c) and (d); (f) manufacturing the collagen fiber constructs or cruciate ligament types “0”, “1”, “2”, “3” and/or “4” described in detail hereinafter; (g) subsequently sterilizing the collagen fiber construct in alcohol; and (h) sterilizing the collagen fiber construct by irradiation.

The isolation of collagen-containing tissue can, according to the invention, comprise individual and/or several ones of the following steps: (a) washing the rat tails with an isotonic/iso-osmolar solution, whereby, in a further special embodiment, the washing is effected in a 0.9% NaCl solution or phosphate buffered saline (PBS), whereby this isotonic or iso-osmolar solution is preferably sterilized; (b) sterilizing the rat tails with alcohol, whereby the sterilizing is preferably carried out with at least 60% alcohol (EtOH). Preferably, sterilizing is carried out with 60%, 65%, 70%, 75%, 80%, 85% or 90% EtOH. In a strongly preferred embodiment, the rat tails are sterilized with 70% EtOH. However, sterilization can also be effected at lower EtOH concentrations such as 45%, 50% or 55%; (c) skinning the tails; and (d) washing the skinned tails with a sterile isotonic/iso-osmolar solution, whereby, in a further special embodiment, the washing is effected in a 0.9% NaCl solution or phosphate buffered saline (PBS), whereby this isotonic or iso-osmolar solution is preferably sterilized.

In conformity with the foregoing, the invention comprises a collagen fiber construct wherein the collagen fiber construct, in a preferred embodiment, is a ligament construct and/or tendon construct. In more strongly preferred fashion, the collagen fiber construct is a cruciate ligament construct.

In a preferred embodiment, the hereinabove described comprises a collagen fiber construct wherein the single collagen fibers are preferably sterilized with at least 60% EtOH. Preferably, sterilizing is carried out with 60%, 65%, 70%, 75%, 80%, 85% or 90% EtOH. In a strongly preferred embodiment, the single collagen fibers are sterilized with 70% EtOH. However, sterilization can also be effected at lower EtOH concentrations such as 45%, 50% or 55%.

In strongly preferred embodiments, the present invention comprises several collagen fiber constructs which will hereinafter be referred to, and described, as “cruciate ligament type 0”, “cruciate ligament type 1”, “cruciate ligament type 2”, “cruciate ligament type 3” and “cruciate ligament type 4”.

Therefore, in conformity with the foregoing, the present invention comprises a collagen fiber construct (“cruciate ligament construct 0”) wherein several single collagen fibers, as described above, are fixed at the ends into a bundle. In a preferred embodiment, the bundle consists here of preferably 20 to 100 single collagen fibers, in more strongly preferred fashion of 50 single collagen fibers. In a further preferred embodiment, several bundles are sewn together at the ends. In a strongly preferred embodiment, the bundles are sewn together at the ends via a so-called “baseball stitch”. The term “baseball stitch”, as described herein, is to be understood as follows: The baseball stitch is a medical stitch technique that is used, inter alia, in fixing cruciate ligament grafts. The ends are joined here with a continuous stitch (FIG. 12). For producing the baseball stitch there is used non-absorbable surgical thread material. For reinforcing the implant, up to 3 cm is provided with a baseball stitch at both ends. The continuous stitch is begun with a puncture from outside at a certain angle. The thread end is prevented from slipping through with a knot or loop. The thread with the needle comes out of the implant from below, runs across the implant and is inserted again on the outer edge. The thread always comes out of the implant again obliquely at the bottom at the same angle. After reaching the end of the implant one goes back again, so that a counter-moving pattern arises.

In a strongly preferred embodiment, preferably 2 to 30 bundles are sewn together, in more strongly preferred fashion 6 bundles.

In a further preferred embodiment, the collagen fiber construct consists of two bundles of preferably 20 to 300 single collagen fibers each, in more strongly preferred fashion of 150 single collagen fibers each, which are sewn together at a certain angle. In a special embodiment, the angle preferably amounts to 20 to 45°.

The present invention not only comprises the above-described sewing together of the above-described linear bundle constructs, however, but also applies to all the collagen fiber constructs presented according to the invention hereinafter, and preferably also to the knitted collagen fiber construct described more precisely hereinafter. The described embodiments thus apply not only to the collagen fiber constructs described specifically above but, mutatis mutandis, to all the described constructs.

In particular, in conformity with the foregoing, the length of the collagen fiber constructs, preferably of the cruciate ligament constructs, preferably amounts to 2.5 to 9.0 cm, and the diameter to 0.6 to 1.0 cm. In a further preferred embodiment, the diameter lies in the range of 0.6 to 1.2 cm. In a more strongly preferred embodiment, the diameter amounts to 0.8 cm. In particular, the collagen fiber construct or cruciate ligament construct should preferably be 2.0 to 7.0 cm long in the patient or in the joint, whereby the length can optionally include portions for anchoring and/or likewise optionally can be increased by further portions for anchoring. In one embodiment, the person skilled in the art can work in one of the following described ranges in order to adapt the length of the collagen fiber construct or cruciate ligament construct in the patient or in the joint, whereby the present invention is not limited to the stated ranges and the person skilled in the art can accordingly choose different ranges and work therein. The depth of the femoral tunnel (normally 1.0 to 3.5 cm, particularly preferably 2.0 cm) and tibial tunnel (normally 1.5 to 4.0 cm, preferably 2.6 cm) is determined using a depth gauge. The intra-articular length is determined individually by the surgeon, normally amounting to between 2.2 and 2.4 cm, particularly preferably between 2.0 and 3.0 cm. The depth gauge used here may be the drill or a measuring rod. The depth gauge is introduced into the tunnel at one end and advanced to the end. The depth gauge possesses either a length scale with which the depth of the tunnel can be directly determined, or the corresponding piece of the depth gauge corresponding to the depth of the tunnel is subsequently measured out. The determination of the depth of the tunnels in the patient is preferably carried out during the cruciate ligament operation.

The above-described embodiment thus applies not only to the collagen fiber constructs described specifically above, but also, mutatis mutandis, to all the constructs described hereinafter and in particular also to the knitted collagen fiber construct described more precisely hereinafter.

As described above, the present invention comprises, in conformity with the foregoing, a further collagen fiber construct (“cruciate ligament construct 1”). Here, several single collagen fibers are preferably knotted into a collagen thread. The knot used therefor can be e.g. a “figure-eight knot” (double loop knot or thumb knot, a simple or half loop, a “square knot” or a triple overhand loop (granny knot, overhand knot) (FIG. 11). The stated knots are described comprehensibly in the literature (Clifford W. Ashley: The Ashley Book of Knots. Over 3800 knots. How they look. What they are used for. How they are made. [German edition] Edition Maritim, Hamburg, 2005. ISBN 3-89225-527-X).

For producing collagen thread, the individual collagen fibers can be knotted together with a thumb knot, an overhand loop or an overhand knot (FIG. 11).

Thumb Knot (Tied without an Object)

In the thumb knot, in a 1st step a loop is laid with the parallel ends of the collagen fibers, so that both ends of the collagen fibers are on top. In a 2nd step the ends of the collagen fibers are pulled through the middle from below, so that the ends are on top again. Then, in steps 3 and 4 the beginnings and ends of the two collagen fibers are respectively pulled carefully, so that the loops increasingly contract and thus yield a knot. Steps 1-4 can be repeated, so that a triple knot arises.

### Overhand Loop

In the overhand loop, in a 1st step a loop is laid with the end of the collagen fiber, so that this piece of the collagen fiber is on top (arrows 1-6). In a 2nd step the end of the collagen fibers is pulled through the middle from below, so that the end is on top again. Then, in steps 3 and 4 the beginning and the end of the collagen fiber are respectively pulled carefully, so that the loops increasingly contract and yield a knot. Steps 1-4 are repeated twice, so that in the end there are three knots lying one over the other.

### Overhand Knot

In a first step 1, two collagen fibers are laid one over the other so as to yield an X. In the 2nd step the collagen fiber (a) on the bottom is laid over the upper collagen fiber (b), and collagen fiber (a) pulled through under collagen fiber (b) again. Then in the 3rd step the beginning of collagen fiber (b) is laid over the end of collagen fiber (a), and in step 4 the end of collagen fiber (b) laid first under and then over collagen fiber (b). Finally, in step 5 the collagen fibers (a) and (b) are carefully pulled in the opposite direction. Steps 3-5 can be repeated, so that a double overhand knot arises.

In a further, strongly preferred embodiment, the present invention comprises a collagen fiber construct wherein the above-described collagen thread or several collagen threads are knitted into a collagen cord. Knitting is preferably done with a knitting spool (see FIGS. 8 to 10). A knitting spool preferably consists of a cylinder with a central bore (tube) which possesses at one end preferably 4 to 8 pins, hooks or the like (cf. FIG. 8) to hold the collagen thread during knitting. For example, a simple knitting spool can be made from a 1 ml syringe (as the tube) and 4 fixing pins (as the pins). A semiautomatic variant is called a “knitting mill”. In particular, upon knitting of the collagen thread into a collagen cord, the collagen thread is first clamped in the knitting spool. In so doing, one end of the collagen thread is threaded through the central bore of the cylinder and held firmly below the cylinder. The part of the collagen thread protruding from the top of the cylinder is wound around the first pin/hook in the counter-clockwise direction, then guided to the left to the second pin/hook and wrapped in the counter-clockwise direction again. These steps are repeated until all the pins are wrapped and thus there is a knitting stitch on each pin/hook (see FIG. 9). All statements regarding the thread guiding can, in a further embodiment, also be reversed, i.e. the pins are respectively wrapped in the clockwise direction. The first pin is then followed by the one adjacent on the right, etc. The actual knitting of the collagen thread is preferably effected by the free end of the collagen thread being tensioned on the outside before the next pin/hook (no. 1) lying on the left (with the reverse arrangement, on the right) of the last (newest) knitting stitch. The collagen thread is, in so doing, tensioned above the knitting stitch lying around this pin/hook (see FIGS. 10 a and b). Subsequently, this knitting stitch is cast inwardly over the new collagen thread and the pin/hook (FIG. 10 c), so that a new knitting stitch comes to lie around the aforesaid pin/hook and the “old” knitting stitch can slip into the central bore of the cylinder (see FIG. 10 d). Next, the free end of the collagen thread is tensioned from outside before pin/hook no. 2 (see FIG. 10 e) in order to produce a new knitting stitch and let the old knitting stitch slide into the central bore there, too, by execution of the above steps. When the stated steps are carried out repeatedly on all pins/hooks, there arises a collagen cord that runs out of the knitting spool downward. In a preferred embodiment, the length of the cord can be freely chosen here. In a further preferred embodiment, the knitting or guiding of the knitting stitches can be facilitated by a needle, curved tweezers or the like and, for finishing, the collagen thread can be guided through one or several of the last knitting stitches and thus knotted and optionally secured by additional knots. It is important here that, besides the collagen threads, or segments of collagen threads, extending in the longitudinal direction of the collagen fiber construct, collagen threads or segments of collagen threads also extend perpendicular to the longitudinal direction of the collagen fiber construct and/or at an angle to the longitudinal direction thereof.

In a further, strongly preferred embodiment, the present invention comprises a collagen fiber construct wherein one or optionally several of the above-described collagen cords (the collagen thread knitted into a collagen cord) are twisted. The term “twist”, as described herein, refers to the winding together and mutual helical wrapping of fibers or wires. When wires are twisted and in telecommunications one also speaks of stranding. Whereby, in connection with the present invention, the term “twist” refers in particular and preferably to the winding together and mutual helical wrapping of collagen cords.

In a further preferred embodiment, the above-described collagen cord is additionally “flipped over”. When being “flipped over”, individual and/or several twisted collagen cords are preferably folded together in the middle. This causes the length to be shortened e.g. to one half, and the collagen cord portions then lying side by side can turn around each other (due to the preceding twisting). Optionally, the collagen cords can be twisted and/or flipped over several times.

In a further, strongly preferred embodiment, the present invention comprises a collagen fiber construct wherein the above-described collagen thread or several collagen threads are coiled, so that several thread portions come to lie parallel to each other. In a preferred embodiment, the thus coiled collagen thread can be used as a collagen fiber construct, in a strongly preferred embodiment as a cruciate ligament construct. The collagen fiber construct can possess an arbitrary, adjustable length. In a strongly preferred embodiment, the length of the collagen fiber construct lies in the range of preferably 2.5 to 9.0 cm and the diameter in the range of preferably 0.6 to 1.0 cm. In a further preferred embodiment, the diameter lies in the range of 0.6 to 1.2 cm. In a more strongly preferred embodiment, the diameter of the collagen fiber constructs manufactured as described above amounts to 0.8 cm. In particular, the collagen fiber construct or cruciate ligament construct should preferably be 2.5 to 7.0 cm long in the patient or in the joint, whereby this length can optionally include portions for anchoring and/or there can likewise optionally be added to this length further portions in order to anchor the collagen fiber construct or cruciate ligament construct.

In a further, strongly preferred embodiment, the collagen fiber construct manufactured as described above can be strengthened at the ends by additional collagen threads and/or collagen fibers.

In a further, strongly preferred embodiment, the collagen fiber construct manufactured as described above can be strengthened at the ends by additional collagen threads or collagen fibers.

As described above, the present invention comprises, in conformity with the foregoing, a further collagen fiber construct (“cruciate ligament construct 2”). Here, individual or several ones of the above-described collagen threads are preferably twisted. Whereby, as described above, the present invention uses the term “twist” in a further embodiment for the winding together and mutual helical wrapping of collagen threads. In a further preferred embodiment, the above-described twisted and/or coiled collagen threads can be “flipped over”. When being “flipped over”, individual and/or several twisted collagen threads are preferably folded together in the middle, causing the length to be shortened e.g. to one half, and the collagen cord portions then lying side by side can turn around each other (due to the preceding twisting). Optionally, the collagen cords can be twisted and/or flipped over several times.

In particular, the above-described cruciate ligament construct (i.e. one manufactured from collagen fibers which was connected into a collagen thread by knotting (“cruciate ligament type 1”) and subsequently knitted into a collagen cord, whereby the cords were subsequently coiled several times and finally twisted (“cruciate ligament type 2”)) surprisingly has a number of advantages over the constructs described in the prior art, as illustrated in the in vivo grafting experiments of the subsequent examples. In particular, these constructs manufactured from pure collagen fibers are characterized by a constant tear strength and a very good incorporation potential, i.e. there are no inflammatory reactions, and the tear strength of the collagen constructs lay in the range of the initial tear strength of the constructs prior to implantation, or could even be increased. Moreover, as surprisingly shown in the examples, the constructs used are accepted well by the bodies of the laboratory animals, for a ligamentization could be observed.

As described above, the present invention comprises, in conformity with the foregoing, a further collagen fiber construct (“cruciate ligament construct 3”). Here, individual or several ones of the above-described collagen threads and/or collagen cords are preferably braided. The term “braid”, as described herein, preferably comprises the regular intertwining of several strands (collagen threads and/or collagen cords) which are thereby guided one over and under the other, so that in the braided state they run around each other in the clockwise and/or counter-clockwise direction. In a special embodiment, three strands can be braided together in particular in the following way (see FIG. 7): (1) three parallel collagen threads and/or collagen cords (=three strands); (2) first lay the left strand (a) over the middle strand (cf. arrow); (3) then lay the right strand (c) over the then middle strand (a) (cf. arrow); (4) then lay the left strand (b) over the strand (c) then lying in the middle again; (5) then lay the right strand (a) over the strand (b) then in the middle again. Points 2, 4 and 3, 5 are repeated until the end of the strands is reached. Alternatively, one can begin from the right with strand (c) in mirror-inverted fashion. Moreover, several collagen threads and/or collagen cords can respectively be combined into a strand. Additionally, the braiding pattern can be transferred to a greater number of strands. In so doing, one proceeds analogously to steps 2 to 5. In a further preferred embodiment, braiding is preferably effected with three to six collagen threads and/or collagen cords which are alternately guided one over the other.

In a further preferred embodiment, the collagen fiber construct can consist of a combination of the above-described embodiments.

As described above, the present invention comprises, in conformity with the foregoing, a further collagen fiber construct (“cruciate ligament construct 4”). Here, the collagen fiber construct is of branched structure. In a preferred embodiment, the collagen fiber construct copies the geometry of a natural tendon or of a natural ligament here with two fiber bundles (consisting of preferably 20 to 300 single collagen fibers each, in more strongly preferred fashion of 150 single collagen fibers each), whereby this collagen fiber construct, in a strongly preferred embodiment, is a cruciate ligament, consisting of two fiber bundles. This cruciate ligament construct is, as described herein, also referred to by the term “double bundle”.

In conformity with the foregoing, the present invention comprises a collagen fiber construct, strongly preferably a cruciate ligament construct, wherein the collagen fiber construct is sterilized with gamma radiation. In particular, the irradiation intensity and dose upon sterilization with gamma radiation can be varied depending on the requirements. In a special embodiment, the irradiation intensity and dose is determined by the German Medicinal Devices Act. In particular, the sterilization for medicinal devices is determined by the sterilization standards DIN EN 550, 552, 556 and DIN EN ISO 17664 valid at the time of filing. In a special embodiment, irradiation is done, depending on the classification, with an energy dose of at least 15 kGy, in a further embodiment with energy doses of at least 15 to 35 kGy, in a strongly preferred embodiment with energy doses of more than 25 kGy, for eliminating germs (bacteria, fungi, viruses). In a more strongly preferred embodiment, there is chosen an irradiation intensity and dose (energy dose) of at least 28.3 kGy. The gamma irradiation is preferably effected with cobalt 60. The cruciate ligament construct, stored in a container (e.g. a 50 ml reaction vessel) filled with buffer solution, is stored in a carton or a Styrofoam box (referred to as the transport box hereinafter) and irradiated analogously to the gamma irradiation of medicinal devices. In so doing, the container is then first loaded into an aluminum container, before being pushed through the irradiation cell with a compressed-air cylinder. Here there is effected, in a preferred embodiment, a gamma irradiation with an energy dose of at least 25 kGy, in a further preferred embodiment there is chosen an irradiation intensity and dose (energy dose) of at least 28 kGy. The measurement of the absorbed energy dose is done using a dosimeter. Advantageously, the transport box did not have to be opened during the gamma irradiation. More exact process data by which the process of irradiation can be effected are to be found in the IAEA guidelines (see also “Trends in radiation of health care products” IAEA (International Atomic Energy Agency) 2008.

In conformity with the foregoing, the present invention comprises a collagen fiber construct wherein the collagen fiber construct, in a strongly preferred embodiment, is an anterior cruciate ligament and/or a posterior cruciate ligament.

The present invention moreover comprises, in conformity with the foregoing, also collagen fiber constructs wherein the collagen fiber constructs are modified by the binding of biomolecules. In a special embodiment, the biomolecules promote ligamentization. Ligamentization is understood to mean a metaplastic process wherein the implant adapts biochemically. This means that cells (primarily fibroblasts) attach to the implant, proliferate, migrate and form a ligamentary (ligament-specific) matrix. Furthermore, endothelial cells immigrate, which lead to vascularization (=formation of blood vessels).

In particular, the present invention also comprises the modification of the collagen fiber constructs which are modified by the binding of biomolecules, wherein the biomolecules preferably induce chemotaxis, cell proliferation, cell migration and/or matrix production. In a strongly preferred embodiment, the biomolecules are selected from the group consisting of chemokines, growth factors, cytokines and active peptides. In particular, in a further strongly preferred embodiment, the biomolecules are selected from the group consisting of platelet-derived growth factor (PDGF), transforming growth factor (TGF), fibroblast growth factor (FGF), bone morphogenic growth factor, bone morphogenic protein (BMP), epidermal growth factor (EGF), insulin growth factor (IGF) and fibronectin; regarding the biomolecules see in particular also Table 1.

In a further preferred embodiment, the collagen fiber construct is to be seeded with fibroblasts and/or epithelial cells on its own in the body after grafting, whereby the seeding can be promoted by the above-described biomolecules. The herein-described modification of the collagen fiber construct by binding of biomolecules will be described further hereinafter:

### Modification of the Collagen Fiber Construct: Binding of Biomolecules

After implantation (FIG. 3) upon a rupture of the anterior cruciate ligament, the cruciate ligament construct is to be seeded by fibroblasts and epithelial cells.

After implantation, the collagen fiber constructs are to be seeded as quickly as possible by cells which then produce a ligament- or tendon-specific extracellular matrix (“ligamentization”).

Besides the use of native collagen fiber constructs, it is possible, as described above, to modify these collagen fiber constructs by biomolecules. The binding of additional biomolecules (chemokines, cytokines) is effected here e.g., but not exclusively, via covalent bonds with collagen fibers. This leads to a chemotaxis and proliferation (of fibroblasts, epithelial cells), cell migration, matrix production (in the adjacent connective tissue) is induced.

Biomolecules such as chemokines, growth factors, cytokines and active peptides can thus promote “ligamentization”.

These biomolecules include (see also Table 1):
Platelet derived growth factor (PDGF-AA, PDGF-AB, PDGF-BB)
Transforming growth factor (TGF-β1 and -β2)
Fibroblast growth factor (FGF-1, FGF-2 and bFGF)
Bone morphogenetic protein (BMP-12 and -13)
Epidermal growth factor (EGF)
Insulin growth factor (IGF)
Fibronectin

TABLE 1
Biomolecules that increase proliferation, matrix production and migration.

| Biomolecule | Effect | Literature |
|---|---|---|
| PDGF-BB/ | Strengthens chemotaxis of fibroblasts | Möller et al., Orthopäde |
| PDGF-AB | Increases proliferation | 29, 182-187 (2000) |
| | Increases collagen type III and V synthesis | Ross et al., Cell 46, 155-169 |
| | Strengthens migration of ligament | (1986) |
| | fibroblasts | Bosch and Krettek, |
| | | Unfallchirurg 105, 88-94 |
| | | (2002) |
| TGF-β1 | Increases collagen synthesis | Bosch and Krettek, |
| | Increases extracellular matrix synthesis | Unfallchirurg 105, 88-94 |
| | | (2002) |
| bFGF | Promotes angiogenesis and proliferation | Cochran and Wozney, |
| | | Periodontology 2000 19, |
| | | 40-58 (1999) |
| BMP-12 | Increases collagen type 1 and elastin | Möller et al., Orthopäde |
| (=GDF-7) | expression | 29, 182-187 (2000) |
| BMP-13 | Increases collagen type 1 and elastin | Möller et al., Orthopäde |
| (=GDF-6) | expression | 29, 182-187 (2000) |
| EFG | Increases collagen synthesis | Bosch and Krettek, |
| | Strengthens migration of ligament | Unfallchirurg 105, 88-94 |
| | fibroblasts | (2002) |
| IGF-1 | Increases proliferation | Cochran and Wozney, |
| | Increased matrix synthesis | Periodontology 2000 19, |
| | | 40-58 (1999) |
| | | Bosch and Krettek, |
| | | Unfallchirurg 105, 88-94 |
| | | (2002) |
| Fibronectin | Increases attachment of cells | Cochran and Wozney, |
| | Increases proliferation | Periodontology 2000 19, |
| | | 40-58 (1999) |

PDGF increases proliferation and stimulates, inter alia, the production of collagen III and V, components of tendons and ligaments (Table 1). The combination of different biomolecules, e.g. PDGF-BB with TGF-β1, can strengthen the effects further.

In a preferred embodiment, the present invention comprises a method for manufacturing a collagen fiber construct composed of single collagen fibers, which is sterilized with alcohol and/or via irradiation and is not populated with cells, wherein the single collagen fibers are isolated from collagen-containing tissue from mammals. In a preferred embodiment, the present invention comprises a method for manufacturing a collagen fiber construct composed of single collagen fibers, which is sterilized with alcohol and via irradiation and is not populated with cells, wherein the single collagen fibers are isolated from collagen-containing tissue from mammals.

The term “collagen-containing tissue” comprises here not only the tissue of mammals and, in a preferred embodiment, that from rat tails. The term also relates to tissue from other organisms and body parts. Thus, the collagen-containing tissue can preferably stem from kangaroos, bovine animals and humans. In a strongly preferred embodiment, the collagen-containing tissue is isolated from rat tails.

In conformity with the foregoing, the invention comprises a method for manufacturing a collagen fiber construct wherein the collagen fiber construct, in a preferred embodiment, is a ligament construct and/or tendon construct. In more strongly preferred fashion, the method is one for manufacturing a collagen fiber construct wherein the collagen fiber construct is a cruciate ligament construct.

In conformity with the foregoing, the present invention comprises a method for manufacturing one of the above-described collagen fiber constructs wherein the isolation and sterilization of the single collagen fibers and the manufacture of the collagen fiber constructs optionally comprises the steps of: (a) isolating collagen-containing tissue; (b) extracting individual and/or several single collagen fibers from the collagen-containing tissue; (c) incubating the single collagen fibers in an isotonic or iso-osmolar solution, whereby, in a further special embodiment, the incubation of the collagen fibers is effected in a 0.9% NaCl solution or phosphate buffered saline (PBS), whereby this isotonic or iso-osmolar solution is preferably sterilized; (d) sterilizing the single collagen fibers in alcohol; (e) optionally repeating the washing and sterilization steps according to points (c) and (d); (f) optionally fixing several isolated and sterilized single collagen fibers into a bundle, this preferably resulting in the above-described collagen fiber construct or the cruciate ligament type “0”; (g) optionally sewing several bundles together at the ends into a collagen fiber construct; (h) sterilizing the collagen fiber construct in alcohol; and (i) sterilizing the collagen fiber construct by irradiation.

In conformity with the foregoing, the present invention comprises a method wherein the described isolation of collagen-containing tissue comprises individual and/or several ones of the following steps: (a) washing the rat tails with an isotonic/iso-osmolar solution, whereby, in a further special embodiment, the washing is effected in a 0.9% NaCl solution or phosphate buffered saline (PBS), whereby this isotonic or iso-osmolar solution is preferably sterilized; (b) sterilizing the rat tails with alcohol, whereby they are sterilized with at least 60% EtOH, in preferred embodiments with 60%, 65%, 70%, 75%, 80%, 85% or 90% EtOH. In a strongly preferred embodiment, the rat tails are sterilized with 70% EtOH. However, sterilization can also be effected at lower EtOH concentrations such as 45%, 50% or 55%; (c) skinning the tails; and (d) washing the skinned tails with a sterile isotonic/iso-osmolar solution, whereby, in a further special embodiment, the washing is effected in a 0.9% NaCl solution or phosphate buffered saline (PBS), whereby this isotonic or iso-osmolar solution is preferably sterilized.

In a further embodiment, the present invention involves a method wherein the collagen fibers, after the extracting from the isolated collagen-containing tissue, are added to a sterile NaCl solution and are sterilized in alcohol. In conformity with the foregoing, the steps of incubating and sterilizing the single collagen fibers are repeated several times in the described method, being repeated three times in a preferred embodiment.

Preferably, there are fixed into a bundle in the above-described method preferably 20 to 100 single collagen fibers, in more strongly preferred fashion 50 single collagen fibers. In a further preferred embodiment, several bundles are sewn together at the ends in the above-described method. In a strongly preferred embodiment, the bundles are sewn together at the ends in the above-described method via a so-called “baseball stitch”. The term “baseball stitch”, as described herein, is to be understood as follows: The baseball stitch is a continuous stitch. For producing the baseball stitch there is used non-absorbable surgical thread material. For reinforcing the implant, up to 3 cm is provided with a baseball stitch at both ends. The continuous stitch is begun with a puncture from outside at a certain angle. The thread end is prevented from slipping through by a knot or loop. The thread with the needle comes out of the implant from below, runs across the implant and is inserted again on the outer edge. The thread always comes out of the implant again at the same angle obliquely at the bottom. After reaching the end of the implant one goes back again, so that a counter-moving pattern arises.

In a strongly preferred embodiment, preferably 2 to 30 bundles are sewn together in the above-described method, in more strongly preferred fashion 6 bundles.

In conformity with the foregoing, the present invention comprises a method for manufacturing a cruciate ligament construct (cruciate ligament type “1”) wherein individual or several single collagen fibers are knotted into a collagen thread.

In a further embodiment, the method for manufacturing a cruciate ligament construct can comprise a method wherein, as described above, individual and/or several collagen threads are knitted into a collagen cord. In further preferred embodiments, individual and/or several collagen cords can be twisted, in the method, and optionally, in a further preferred embodiment, flipped over, as described above. In particular, these steps can be carried out several times in succession when required.

In conformity with the foregoing, the present invention comprises a method for manufacturing a cruciate ligament construct (cruciate ligament type “2”) wherein, in this method, individual or several collagen threads are twisted as described above. In further preferred embodiments, the twisted collagen threads can be flipped over, in a further preferred embodiment, as described above.

In conformity with the foregoing, the present invention comprises a method for manufacturing a cruciate ligament construct wherein, in this method, individual or several collagen threads are coiled, so that several thread portions come to lie parallel to each other. In a preferred embodiment, the thus coiled collagen thread can be used as a collagen fiber construct, in a strongly preferred embodiment, as a cruciate ligament construct. The collagen fiber construct here can possess an arbitrary, adjustable length. In a strongly preferred embodiment, the length of the collagen fiber construct lies in the range of preferably 2.5 to 9.0 cm and the diameter in the range of preferably 0.6 to 1.0 cm. In a further preferred embodiment, the diameter lies in the range of 0.6 to 1.2 cm. In a more strongly preferred embodiment, the diameter of the collagen fiber constructs manufactured according to the method amounts to 0.8 cm. In particular, the collagen fiber construct or cruciate ligament construct should preferably be 2.5 to 7.0 cm long in the patient or in the joint, whereby this length can optionally include portions for anchoring and/or there can likewise optionally be added to this length further portions in order to anchor the collagen fiber construct or cruciate ligament construct.

In a further, strongly preferred embodiment, the collagen fiber construct manufactured according to the method can be strengthened at the ends by additional collagen threads and/or collagen fibers.

In a further, strongly preferred embodiment, the collagen fiber construct manufactured according to the method can be strengthened at the ends by additional collagen threads or collagen fibers.

In conformity with the foregoing, the present invention moreover comprises a method for manufacturing a cruciate ligament construct (cruciate ligament type “3”) wherein, in this method, individual or several ones of the above-described cruciate ligament threads and/or collagen cords are braided as stated above. The braiding is preferably effected here with three to six collagen threads and/or collagen cords which, as described above, are guided alternately one over the other. Preferably, the braiding is effected as described above and as illustrated in FIG. 7.

In a further embodiment, the above-described methods can be carried out several times in succession and/or be combined with each other.

In conformity with the foregoing, the present invention moreover comprises a method for manufacturing a cruciate ligament construct (cruciate ligament type “4”) wherein the collagen fiber construct is of branched structure. Preferably, the collagen fiber construct is so structured in this method that the collagen fiber construct copies the geometry of a natural tendon or of a natural ligament here with two fiber bundles (consisting of preferably 20 to 300 single collagen fibers each, in more strongly preferred fashion of 150 single collagen fibers each), whereby this collagen fiber construct, in a strongly preferred embodiment, is a cruciate ligament, consisting of two fiber bundles. This cruciate ligament construct according to the invention manufactured by this method is, as described herein, also referred to by the term “double bundle”.

In conformity with the foregoing, the present invention comprises a method for manufacturing a collagen fiber construct, strongly preferably a cruciate ligament construct, wherein the collagen fiber construct is sterilized in this method with gamma radiation, as described above. In particular, the irradiation intensity and dose (energy dose) upon sterilization with gamma radiation can be varied depending on the requirements, as described above. Preferably, the irradiation is effected in this method with an energy dose of at least 28.3 kGy. In a special embodiment, irradiation is done with an energy dose of at least 15 kGy, in a further embodiment with energy doses of at least 15 to 35 kGy, in a strongly preferred embodiment with energy doses of more than 25 kGy. In a more strongly preferred embodiment, there is chosen an irradiation intensity and dose (energy dose) of at least 28.3 kGy. As described above, a gamma irradiation can be effected in a preferred embodiment with an energy dose of at least 25 kGy, in a strongly preferred embodiment with an irradiation intensity and dose (energy dose) of at least 28.3 kGy.

Preferably, the present invention involves a method for manufacturing a collagen fiber construct, strongly preferably a cruciate ligament construct, wherein the above-described incubating and washing steps according to the method are preferably effected in an isotonic or iso-osmolar solution, whereby the incubating and washing steps, in a further special embodiment, are effected in a 0.9% NaCl solution or phosphate buffered saline (PBS). This isotonic or iso-osmolar solution is preferably sterilized. In a further embodiment of the method, the above-described sterilizing steps according to the method are preferably effected with at least 60% EtOH, preferably with 60%, 65%, 70%, 75%, 80%, 85% or 90% EtOH. In a strongly preferred embodiment, the sterilizing steps are carried out with 70% EtOH. However, sterilization can also be effected at lower EtOH concentrations such as 45%, 50% or 55%.

Preferably, in the herein-described method, the length of the collagen fiber constructs, preferably of the cruciate ligament constructs, is chosen such that it amounts to preferably 2.5 to 9.0 cm and the diameter preferably 0.6 to 1.0 cm. In a further preferred embodiment, the diameter lies in the range of 0.6 to 1.2 cm. In a more strongly preferred embodiment, the diameter of the collagen fiber constructs manufactured according to the method amounts to 0.8 cm. In particular, the collagen fiber construct or cruciate ligament construct should preferably be 2.5 to 7.0 cm long in the patient or in the joint, whereby the collagen fiber construct or cruciate ligament construct can optionally include portions for anchoring and/or there can likewise optionally be added to the length further portions for anchoring. In one embodiment, the person skilled in the art can work in one of the following described ranges in order to adapt the length of the collagen fiber construct or cruciate ligament construct in the patient or in the joint, whereby the present invention is not limited to the stated ranges and the person skilled in the art can accordingly choose different ranges and work therein. The depth of the femoral tunnel (normally 1.0 to 3.5 cm, particularly preferably 2.0 cm) and tibial tunnel (normally 1.5 to 4.0 cm, preferably 2.6 cm) is determined using a depth gauge. The intra-articular length is determined individually by the surgeon, normally amounting to between 2.2 and 2.4 cm, particularly preferably between 2.0 and 3.0 cm. The depth gauge used here may be the drill or a measuring rod. The depth gauge is introduced into the tunnel at one end and advanced to the end. The depth gauge possesses either a length scale with which the depth of the tunnel can be directly determined, or the corresponding piece of the depth gauge corresponding to the depth of the tunnel is subsequently measured out. The determination of the depth of the tunnels in the patient is preferably carried out during the cruciate ligament operation.

The present invention moreover comprises, in conformity with the foregoing, also a method for manufacturing collagen fiber constructs wherein the collagen fiber constructs are modified by the binding of biomolecules. In a special embodiment, the biomolecules promote ligamentization. Ligamentization is understood to mean a metaplastic process wherein the implant adapts biochemically. This means that cells (primarily fibroblasts) attach to the implant, proliferate, migrate and form a ligamentary matrix. Furthermore, endothelial cells immigrate, which lead to vascularization (=formation of blood vessels).

In particular, the method of the present invention also comprises the modification of the collagen fiber constructs which are modified by the binding of biomolecules, wherein the biomolecules preferably induce chemotaxis, cell proliferation, cell migration and/or matrix production. In a strongly preferred embodiment, the biomolecules of the above-described method are selected from the group consisting of chemokines, growth factors, cytokines and active peptides. In particular, in a further strongly preferred embodiment, the biomolecules are selected from the group consisting of platelet-derived growth factor, transforming growth factor, fibroblast growth factor, bone morphogenic growth factor, epidermal growth factor, insulin growth factor and fibronectin (Table 1). In a further preferred embodiment, the method comprises the manufacture of a collagen fiber construct which is seeded with fibroblasts and/or epithelial cells on its own in the body after implantation, whereby the seeding can be promoted by the above-described biomolecules.

Finally, the present invention comprises a collagen fiber construct manufacturable or manufactured by one of the above-described methods. The embodiments which are disclosed in connection with the method of the present invention also apply, mutatis mutandis, to the collagen fiber construct manufacturable or manufactured by one of the above-described methods. The collagen fiber construct manufacturable or manufactured by one of the above-described methods is preferably a tendon construct and/or ligament construct, in a strongly preferred embodiment a cruciate ligament construct.

Moreover, the present invention comprises the above-described collagen fiber construct for use in the treatment of orthopedic diseases and/or as a xenoimplant. The embodiments which are described and disclosed above in connection with the method of the present invention and with the collagen fiber construct of the present invention also apply, mutatis mutandis, to use in the treatment of orthopedic diseases and/or as a xenoimplant. In a further embodiment, the present invention comprises the above-described collagen fiber construct wherein the orthopedic disease is a cruciate ligament rupture. In further embodiments, the present invention comprises the above-described collagen fiber construct wherein the orthopedic disease is an Achilles tendon rupture.

Furthermore, the orthopedic disease can be an injury and/or degeneration of the tendons of the rotator cuff (shoulder). Also, the orthopedic disease can be an injury/rupture of the lateral collateral ligaments on the knee or on the ankle. The orthopedic disease can also be an injury/rupture or degeneration of the medial patellofemoral ligament (MPFL). In a strongly preferred embodiment, the collagen fiber construct for use in the treatment of orthopedic diseases and/or as a xenoimplant is a cruciate ligament construct. In conformity with the foregoing, the collagen fiber construct for use in the treatment of orthopedic diseases and/or as a xenoimplant can be an Achilles tendon construct, a tendon construct of the rotator cuff, a construct of the lateral collateral ligaments on the knee or on the ankle, or a construct of the MPFL.

Advantageously, the present invention comprises the use of the above-described collagen fiber construct as a xenoimplant. In a further preferred embodiment, the above-described collagen fiber construct can be used as a xenograft and/or implant or graft composed of human collagen. The embodiments which are described and disclosed above in connection with the method of the present invention and with the collagen fiber construct of the present invention also apply, mutatis mutandis, to the use as a xenoimplant, xenograft, implant or graft composed of human collagen. In a strongly preferred embodiment, the use of the present invention is the use of a tendon construct and/or ligament construct, whereby, in a further strongly preferred embodiment, this is a cruciate ligament construct.

Moreover, the present invention comprises a container which contains the above-described collagen fiber constructs, preferably cruciate ligament constructs, in a suitable solution. Preferably, the solution involves an isotonic/iso-osmolar solution, whereby, in a further special embodiment, the storage and/or the transport of the constructs in this container is effected in a 0.9% NaCl solution or phosphate buffered saline (PBS), whereby this isotonic or iso-osmolar solution is preferably sterilized. The constructs can be stored and/or transported in an above-described solution in order to avoid exsiccation of the constructs.

### Connecting of Several Single Fibers

In some cases the tear strength can lie below the theoretical tear strength of the cruciate ligament construct. This is due to the different length and pre-tension of the single fibers used, i.e. as of a certain tension the shortest fibers always break successively, since they must quasi carry the total force alone.

The term tear strength describes here the tensile force (the unit being the newton=N) at which the collagen fiber construct breaks upon tensile load. The tear strength per unit area (the unit being N/mm²) describes the ratio of the tear strength of a collagen fiber construct to the cross-sectional area of this collagen fiber construct, to permit comparison of different collagen fiber constructs with each other.

A modified construct of the present invention is, in a preferred embodiment, therefore so structured that the applied force is automatically distributed over all the fibers, i.e. there can be effected a compensation of length and/or force between the individual fibers or substructures of the constructs. The distribution of the force can take place uniformly or non-uniformly.

The (re-)distribution of the force applied to a fiber over the adjacent fibers or the construct as a whole can be effected in different ways. A flexible integration of the single fibers into the construct, so that the single fibers still possess a certain mobility in the construct (e.g. shifting for compensating forces), can be advantageous here. Concretely, there are utilized, as described above, in preferred embodiments of the invention the following possibilities, which were verified in simple experiments and led to a clear improvement in tear strength:
Connecting the single fibers by action of mechanical force (e.g. pressing together, raveling out and then connecting)
Connecting the single fibers by thermal treatment (hot and/or cold)
Connecting the single fibers by chemical reaction with or without the use of chemicals (e.g. by partly dissolving the collagen structure and then resolidifying with or without the use of a further chemical reaction)
Connecting the single fibers by biological reaction (e.g. a growing together of individual fibers/strands)
Bonding the single fibers with a suitable “adhesive” (e.g. fibrin adhesive)
Knotting the single fibers
Entwining/Intertwining the single fibers (examples thereof are “knitting with a knitting spool” or “knitting”, “crocheting” in general
Interweaving the single fibers
Braiding the single fibers
Turning/twisting the single fibers

The stated possibilities can be applied here, in conformity with the foregoing, to fibers with the same cross section and/or to fibers of different cross section, e.g. for connecting a fiber with a cross section greater than 0.25 mm² to a fiber with a cross section smaller than 0.25 mm².

The stated possibilities can be applied here, as described above, respectively to individual single fibers and/or a bundle of single fibers. They can also be utilized to connect single fibers to a bundle of single fibers.

Moreover, one possibility can be applied several times in succession, in order to thereby e.g. increase the tear strength successively.

An individual step can definitely reduce the tear strength here (e.g. due to a higher proportion of transverse forces).

Furthermore, different possibilities can be combined with each other and/or executed successively. An example thereof is manufacturing a long fiber by e.g. knotting. This long fiber is subsequently turned, braided, knitted, etc., into a more stable construct. A further example is the combination of turned and braided strands.

Therefore, the present invention relates to collagen fiber constructs, and, in a preferred embodiment, cruciate ligament constructs, which are defined by different tear strengths or tear strengths per unit area. The definition of tear strength is stated above. The tear strength can be determined by subjecting the collagen fiber construct to tensile load. For this purpose, the collagen fiber construct is clamped at both ends. While one end is held firmly, the other end is continuously pulled. In so doing, the tensile force is continuously increased, starting out from a defined tensile force of e.g. 0N. The tensile force is continuously measured. The tensile force at which the collagen fiber construct, or a part of the collagen fiber construct, breaks is equal to the tear strength of the collagen fiber construct.

The tear strength of a natural cruciate ligament lies in the range of 800 to 1800 N, depending, inter alia, on the person's age, sex and weight. The maximum tear strength exists at the age of approx. 22 years in men (Woo, et al., Am. J. Sports Med. 27, 533-543 (1999)).

As was explained more closely in the examples, different tear strengths per unit area were measured in the hitherto tested collagen fiber constructs:

“Cruciate ligament construct 0”: (parallel single fibers)
16 N/mm², at a diameter of the construct of 8 mm this yields a tear strength of approx. 803N.
“Cruciate ligament construct 1” (collagen thread)
31 N/mm², at a diameter of the construct of 8 mm this yields a tear strength of 1557 N.
“Cruciate ligament construct 1” (collagen thread coiled)
28 N/mm², at a diameter of the construct of 8 mm this yields a tear strength of 1406 N.
“Cruciate ligament construct 1” (collagen cord)
60 N/mm², at a diameter of the construct of 8 mm this yields a tear strength of 3014 N.
“Cruciate ligament construct 2” (collagen thread twisted)
58 N/mm², at a diameter of the construct of 8 mm this yields a tear strength of 2913 N.
“Cruciate ligament construct 3” (collagen thread braided)
19 N/mm², at a diameter of the construct of 8 mm this yields a tear strength of 954 N.
“Cruciate ligament construct 4” (branched collagen fiber construct)
Tear strength of the partial strands dependent on the embodiment, see above.

### Construct of Arbitrarily Adjustable Length

Furthermore, there is described herein the manufacture of a construct according to the invention with a freely selectable length in order to make it possible to use the produced constructs for other applications. These include, inter alia, e.g. the use as an Achilles tendon replacement, as a ligament/tendon replacement in the elbow joint or in the shoulder (inter alia, rotator cuff) and the application in domestic and utility animals, e.g. (race) horses.

By the above-mentioned connecting of single fibers there can also be manufactured a construct of arbitrarily adjustable length. This construct can be individually coordinated with different applications and is no longer limited to the pure cruciate ligament construct for use in humans. Further potential areas of application are, as described above, e.g. the use as an Achilles tendon replacement, as a ligament/tendon replacement in the elbow joint or in the shoulder (inter alia, rotator cuff) and the application in domestic and utility animals, e.g. (race) horses, dogs.

In so doing, a parallel arrangement of several strands of the produced construct can again be performed in order to further increase the tear strength. In contrast to the single fibers of limited length, a sufficiently long strand can be guided back and forth between the points of suspension several times here. It is thus possible to compensate length and thus force between the individual portions of the strand.

### Anchoring of the Constructs

Moreover, the anchoring of the above-described (cruciate ligament) constructs of the invention can be realized, e.g. with so-called EndoButtons already used hitherto in cruciate ligament operations. An EndoButton is understood to mean a titanium button/plate with four holes through which the tendon grafts or implants can be drawn and then fixed.

Surgical technique: Prior to incorporation of the construct, the tibial and femoral tunnels are created at the attachment sites of the original cruciate ligament. Subsequently, the graft is stitched up with special thread material and a small plate (EndoButton), and drawn into the joint through two tunnels. The titanium EndoButton is flipped at the upper end and thus holds the construct on the femur. The fixation of the construct on the lower leg is effected either via a small titanium disk (suture disk) or with a screw/dowel.

Altogether it is thereby also possible to realize large-area anchorings which possess a better force-per-area ratio than the hitherto used parallel anchoring of the single fibers. Thus, a higher tear strength of the construct anchored in the bone can be obtained.

Furthermore, it is thereby also possible to realize constructs according to the invention in different forms which can also be fastened at more than two anchoring points. Thus, it is e.g. possible to recreate the form of a natural cruciate ligament which divides into different bundles (see FIG. 5).

### Composite Materials

An additional possibility for manufacturing a stable tendon construct or ligament construct of the above-described embodiments consists in the combination of the collagen fibers and/or collagen constructs with other materials. It is e.g. possible here to increase the basic stability by using silk fibers.

The additional materials can be connected with each other and/or the collagen fibers and/or the collagen constructs using the above-described possibilities (see Connecting of several single fibers).

In addition or as an alternative, one material can enclose the other material or materials, e.g. the connected collagen fibers can be enclosed by a sheath of silk fabric, or a silk strand can be enclosed by a tubularly arranged construct composed of collagen fibers.

The thus manufactured composite construct can in turn be processed further by the possibilities described for connecting the single fibers, and/or be anchored as described above.

### Further Applications

The above-described collagen fiber constructs of the present invention are not limited only to cruciate ligaments (anterior and posterior cruciate ligaments), but can be used as a replacement for all tendons and ligaments (e.g. Achilles tendon, in the shoulder, inter alia, rotator cuff, medial and lateral collateral ligaments, medial patellofemoral ligament, patellar tendon, etc.).

The above-described collagen fiber constructs of the present invention are not limited only to use in humans, but can also be used for tendon and ligament replacement in small and large animals (e.g. dogs, horses, camels, bovine animals, etc.).

### Additional Isolation Source

The collagen fibers of the above-described invention can be obtained not only from rat tails but also from other animals, e.g. from kangaroo tails, bovine tails, dog tails, squirrel tails, pig tendons, bovine tendons. Moreover, the collagen fibers can also be obtained from human tissue.

### Manufacture of Implants and Implantation

In conformity with the foregoing, all the different types can quite generally be manufactured and used in the manufacture of cruciate ligament constructs for use in humans. Thus, all the different types of cruciate ligament construct described can in principle be used. For example, there can be manufactured a cruciate ligament construct wherein collagen fibers are connected by a knot to produce a collagen thread. Individual or several collagen threads can then be coiled, so that several thread portions come to lie parallel to each other. The collagen fiber construct here can possess an arbitrary, adjustable length. For use in humans, the length lies in the range of preferably 2.5 to 9.0 cm and the diameter in the range of preferably 0.6 to 1.0 cm. In a further preferred embodiment, the diameter lies in the range of 0.6 to 1.2 cm. The cruciate ligament construct should preferably be 2.5 to 7.0 cm long in the patient or in the joint (depending on age, sex and physique). Sufficient portions for anchoring might already be included therein or must be added, depending on the method with which the collagen fiber construct is anchored. Suitable methods are commonly known to the person skilled in the art and described in the prior art. As described above, the collagen fiber construct can be strengthened at the ends by additional collagen threads and/or collagen fibers. In so doing, it is e.g. possible to protect the construct from abrasion by a simple wrapping of the ends with collagen threads. A stitching up of the ends of the construct with a collagen thread (e.g. cross stitch or baseball stitch) permits a mechanical stabilization of the construct ends.

Hereinafter there will be described the surgical technique with which the implants were implanted in the miniature pig, as described in the examples. It is commonly known to the person skilled in the art, however, that the surgical technique used can be transferred to humans in minimally invasive form. Suitable methods and techniques are known to the person skilled in the art and described in the prior art. The cruciate ligament constructs are guided through bores in femur and tibia, fastened e.g. with an EndoButton, suture button or steel pin (cruciate ligament anchor). In principle, all known and clinically used variants of cruciate ligament anchors can be used here. In the miniature pigs there were used so-called surgical loops (fabric tapes) to connect the collagen fiber constructs on both sides with the suture buttons (the cruciate ligament constructs were implanted in the miniature pig using suture buttons from Arthrex). In so doing, a fine adjustment of the total length of anchor/button-surgical loop-construct-surgical loop-anchor/button can be performed by the choice of the suitable length of the surgical loops. Moreover, it is important that there is respectively a sufficiently long piece of the collagen construct in the bone tunnels (femur and tibia) to make it possible for the constructs to engraft into the bones. Suitable lengths here are e.g. 1.5 to 4 cm on each side in humans and 1.0 to 2.5 cm in miniature pigs.

### THE FIGURES SHOW

FIG. 1: Isolation of collagen fibers from rat tail. (A) Rat tail; (B) Skinned rat tail; (C) Isolated collagen fiber; (D) Comparison of collagen fiber and skinned rat tail.

FIG. 2: Collagen fiber-based cruciate ligament construct. The construct consists of six collagen fiber bundles with 50 single fibers each, which are sewn together at the ends with a so-called baseball stitch. The length amounts to approx. 7 cm, the diameter is 8 mm.

FIG. 3: Anterior cruciate ligament (ACL) and posterior cruciate ligament (PCL) in the knee. The cruciate ligament construct is used e.g. for a torn ACL.

FIG. 4: “Collagen cord”: Collagen fiber construct manufactured with a knitting spool. Represented is a collagen cord that was manufactured with a knitting spool having four pins. The manufacturing process is described in detail in connection with FIGS. 8-10. There can clearly be seen the V-shaped structure of the individual knitting stitches of the collagen cord. Upon manufacture, a simple collagen thread was used and a cord with a length of approx. 14 cm was manufactured. This collagen cord can be used directly or be processed further (e.g. by braiding with other collagen cords, see FIG. 7).

FIG. 5: Schematic representation of a construct with a dividing structure. This makes it possible to anchor the construct at several fastening points (here two and three, respectively).

FIG. 6: Modification of the collagen fiber constructs with biomolecules. Biomolecules support a faster immigration and adhesion of cells. This achieves a faster ligamentization of the construct used.

FIG. 7: Braiding of collagen threads and/or collagen cords. Upon braiding, several strands (collagen threads and/or collagen cords) are preferably intertwined regularly, being thereby guided one over and under the other, so that in the braided state they run around each other in the clockwise and/or counter-clockwise direction. In so doing, three strands can for example be braided together in the following way: (1) three parallel collagen threads and/or collagen cords (=three strands); (2) first lay the left strand (a) over the middle strand (cf. arrow); (3) then lay the right strand (c) over the then middle strand (a) (cf. arrow); (4) then lay the left strand (b) over the strand (c) then lying in the middle again; (5) then lay the right strand (a) over the then middle strand (b) again. Points 2, 4 and 3, 5 are repeated until the end of the strands is reached. Alternatively, one can begin from the right with strand (c) in mirror-inverted fashion. Moreover, several collagen threads and/or collagen cords can respectively be combined into a strand. Additionally, the braiding pattern can be transferred to a greater number of strands. In so doing, one proceeds analogously to steps 2 to 5.

FIG. 8: Knitting of collagen threads into a collagen cord with the knitting spool—Structure of a knitting spool. A knitting spool preferably consists of a cylinder with a central bore (tube) which possesses at one end preferably four to eight pins, hooks or the like for holding the collagen thread during knitting.

FIG. 9: Knitting of collagen threads into a collagen cord with the knitting spool—Clamping of the collagen thread. Thread one end of the collagen thread through the central bore of the cylinder and hold it firmly below the cylinder. Wind the part of the collagen thread protruding from the top of the cylinder around the first pin/hook in the counter-clockwise direction, then go left to the second pin/hook and wrap in the counter-clockwise direction again. Repeat these steps until all the pins are wrapped and thus there is a knitting stitch on each pin/hook.

FIG. 10: Knitting of collagen threads into a collagen cord with the knitting spool—Knitting of the collagen thread. The actual knitting of the collagen thread is effected by the free end of the collagen thread being tensioned on the outside before the next pin/hook (no. 1) lying on the left (with the reverse arrangement, on the right) of the last (newest) knitting stitch. The collagen thread is, in so doing, tensioned above the knitting stitch lying around this pin/hook ((a) and (b)). Subsequently, this knitting stitch is cast inwardly over the new collagen thread and the pin/hook (c), so that a new knitting stitch comes to lie around the aforesaid pin/hook and the “old” knitting stitch can slip into the central bore of the cylinder (d). Next, the free end of the collagen thread is tensioned from outside before pin/hook no. 2 (e) in order to produce a new knitting stitch and let the old knitting stitch slide into the central bore there, too, by execution of the above steps. When the stated steps are carried out repeatedly on all pins/hooks, there arises a collagen cord that runs out of the knitting spool downward. The length of the cord can be freely chosen.

FIG. 11: Illustration of a thumb knot, an overhand loop and an overhand knot. For producing collagen threads, the individual collagen fibers can be knotted together with a thumb knot, an overhand loop or an overhand knot (FIG. 11).

FIG. 12: Illustration of a baseball stitch. The baseball stitch is a continuous stitch. For producing the baseball stitch there is used non-absorbable surgical thread material. For reinforcing the implant, up to 3 cm is provided with a baseball stitch at both ends. The continuous stitch is begun with a puncture from outside at a certain angle. The thread end is prevented from slipping through by a knot or loop. (1) The thread with the needle comes out of the implant from below, runs across the implant and is inserted again on the outer edge. The thread always comes out of the implant again at the same angle obliquely at the bottom. (2) After reaching the end of the implant one goes back again, so that a counter-moving pattern arises.

### EXAMPLES

### Example 1

### Isolation and Sterilization of Collagen Fibers

The cruciate ligament constructs of the invention are composed of single collagen fibers.

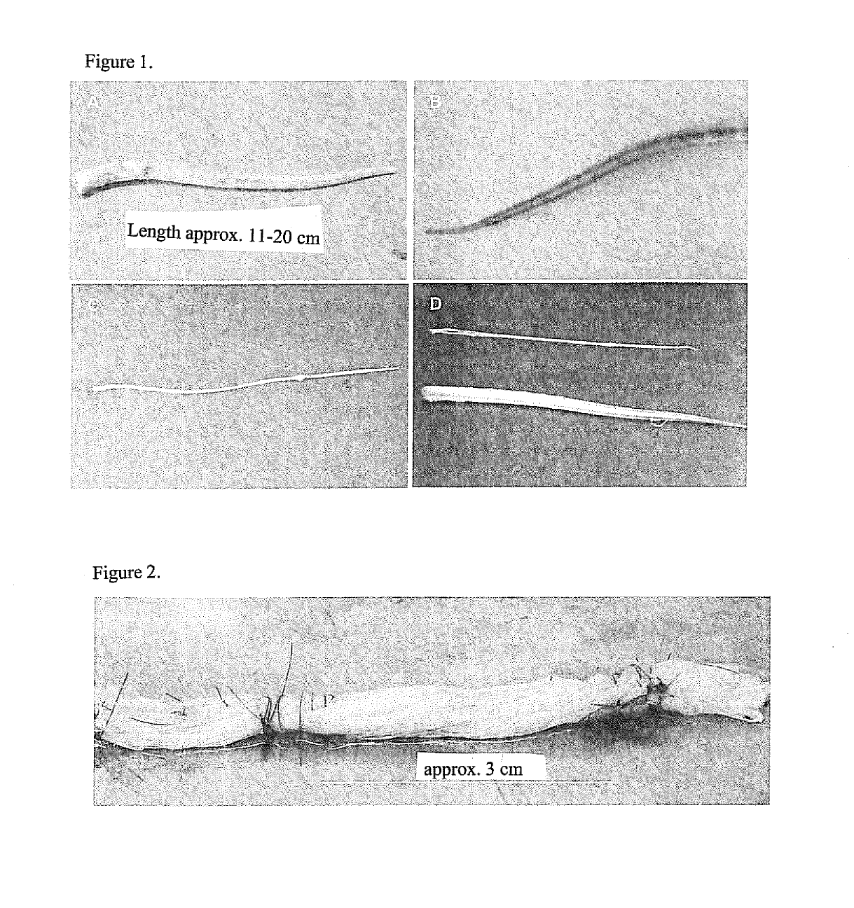

The single collagen fibers are obtained from the tails of rats (FIG. 1). For this purpose, the rat tails are washed with a sterile 0.9% saline solution (0.9% NaCl; pH 7.4, 290 mOsm), sterilized with 70% EtOH for 10 min and carefully skinned. The skinned tails are washed again with 0.9% NaCl solution (pH 7.4, 290 mOsm). The individual collagen fibers are carefully extracted, added to 0.9% NaCl solution (pH 7.4, 290 mOsm) and sterilized again with 70% EtOH for 10 min. The washing and sterilization steps are carried out thoroughly three times altogether. Then the collagen fibers are stored in 0.9% NaCl solution (pH 7.4, 290 mOsm). These sterile collagen fibers can now be used for manufacturing the cruciate ligament constructs.

### Example 2

### Manufacture of the Cruciate Ligament Constructs

For manufacturing the cruciate ligament constructs, in one possibility, fifty single collagen fibers are always arranged parallel and fixed at the ends with a thread into a bundle and then six bundles of fifty are sewn together at the ends with a so-called baseball stitch. The baseball stitch is a continuous stitch. For producing the baseball stitch there is used non-absorbable surgical thread material. For reinforcing the implant, up to 3 cm is provided with a baseball stitch at both ends. The continuous stitch is begun with a puncture from outside at a certain angle. The thread end is prevented from slipping through by a knot or loop. (1) The thread with the needle comes out of the implant from below, runs across the implant and is inserted again on the outer edge. The thread always comes out of the implant again at the same angle obliquely at the bottom. (2) After reaching the end of the implant one goes back again, so that a counter-moving pattern arises (FIG. 2).

A further possibility is to sew together two bundles of 150 single collagen fibers each at a certain angle (approx. 20 to 45°).

The length and the diameter of the cruciate ligament constructs are based on the hitherto used cruciate ligament grafts and amount to 7×0.8 cm. Of this, 2 cm is respectively required at the ends for the baseball stitch or for the anchoring in the bone, so that in the middle the cruciate ligament has an effective length of 3 cm. As the final sterilization method there is effected the gamma irradiation in order to guarantee asepsis (see Example 3). The gamma irradiation is preferably effected with cobalt 60. The cruciate ligament construct, stored in a container (e.g. a 50 ml reaction vessel) filled with buffer solution, is stored in a carton or Styrofoam box (referred to hereinafter as the transport box) and irradiated analogously to the gamma irradiation of medicinal devices. In so doing, the container is then first loaded into an aluminum container before being pushed through the irradiation cell with a compressed-air cylinder. Here there is effected, in a preferred embodiment, a gamma irradiation with a dose (energy dose) of at least 25 kGy. The measurement of the absorbed energy dose is done using a dosimeter. Advantageously, the transport box did not have to be opened during the gamma irradiation. More exact process data by which the process of irradiation can be effected are to be found in the IAEA guidelines (see also “Trends in radiation of health care products” IAEA (International Atomic Energy Agency) 2008.

### Example 3

### Sterilization of Cruciate Ligament Constructs

After manufacture of the collagen fiber constructs, they are sterilized again with 70% EtOH, stored in sterile 0.9% NaCl solution (pH 7.4, 290 mOsm) and finally gamma irradiated (energy dose at least 28.3 kGy).

### Example 4

### Tear Strength

### Sequence of Determination of Tear Strength of the Constructs

### Material Testing

The tear strength can be determined by subjecting the collagen fiber construct to tensile load. For this purpose, the collagen fiber construct is clamped at both ends. While one end is held firmly, the other end is continuously pulled. In so doing, the tensile force is continuously increased, starting out from a defined tensile force of e.g. 0 N. The tensile force is continuously measured. The tensile force at which the collagen fiber construct, or a part of the collagen fiber construct, breaks is equal to the tear strength of the collagen fiber construct.

In an experiment with 300 single collagen fibers, a tear strength per unit area of 16 N/mm² was measured, at a diameter of the construct of 8 mm this yields a tear strength of approx. 803 N.

### Example 5

### Alternative Variants of Manufacturing the Cruciate Ligament Constructs

Alternatively to the method described in Example 3, the cruciate ligament constructs are manufactured in two further different ways, which will hereinafter be referred to as “cruciate ligament type 1” and cruciate ligament type 2”:

To increase the tear strength of the collagen fiber constructs in comparison to the constructs composed of parallel arranged single collagen fibers (see Examples 1 to 4), modified constructs were manufactured.

The tear strength of parallel arranged single collagen fibers lies below the tear strength theoretically computed on the basis of the number of single collagen fibers used. This is due to the different length and pre-tension of the single fibers used, i.e. as of a certain tension the shortest fibers always break successively, since they must quasi carry the total force alone.

A modified construct is therefore so structured that the applied force is automatically distributed over all the fibers, i.e. there can be effected a compensation of length and/or force between the individual fibers or substructures of the constructs. The distribution of the force can take place uniformly or non-uniformly.

The (re-)distribution of the force applied to a fiber over the adjacent fibers or the construct as a whole can be effected in different ways. A flexible integration of the single fibers into the construct, so that the single fibers still possess a certain mobility in the construct (e.g. shifting for compensating forces), can be advantageous here, but need not necessarily be realized. Concretely, the following possibilities are utilized which were partly already verified in experiments and led to a clear improvement in tear strength (see below):
Connecting the single fibers by action of mechanical force (e.g. pressing together, raveling out and then connecting)
Connecting the single fibers by thermal treatment (hot and/or cold)
Connecting the single fibers by chemical reaction with or without the use of chemicals (e.g. by partly dissolving the collagen structure and then resolidifying with or without the use of a further chemical reaction)
Connecting the single fibers by biological reaction (e.g. a growing together of individual fibers/strands)
Bonding the single fibers with a suitable “adhesive” (e.g. fibrin adhesive)
Knotting the single fibers
Entwining/Intertwining the single fibers (examples thereof are “knitting with a knitting spool” or “knitting”, “crocheting” in general
Interweaving the single fibers
Braiding the single fibers
Turning/twisting the single fibers

The stated possibilities can be applied here to fibers with the same cross section and/or to fibers of different cross section, e.g. for connecting a fiber with a cross section greater than 0.25 mm² to a fiber with a cross section smaller than 0.25 mm².

The stated possibilities can be applied here respectively to individual single fibers and/or a bundle of single fibers. They can also be utilized to connect single fibers to a bundle of single fibers.

Moreover, one possibility can be applied several times in succession, in order to thereby e.g. increase the tear strength successively.

An individual step can definitely reduce the tear strength here (e.g. due to a higher proportion of transverse forces).

Furthermore, different possibilities can be combined with each other and/or executed successively. An example thereof is manufacturing a long fiber by e.g. knotting. This long fiber is subsequently turned, braided, knitted, etc., into a more stable construct. A further example is the combination of turned and braided strands.

In particular, the following possibilities were utilized, which were verified in experiments and led to a clear improvement in tear strength. The cruciate ligament constructs are manufactured here in two further different ways, which will be referred to hereinafter as “type 1” and “type 2”:

“Cruciate ligament type 1”: Here, “threads” are produced by knotting the individual collagen fibers. These threads are subsequently knitted into a “collagen cord” with a knitting spool (see FIG. 4).

### Knotting of Collagen Fibers

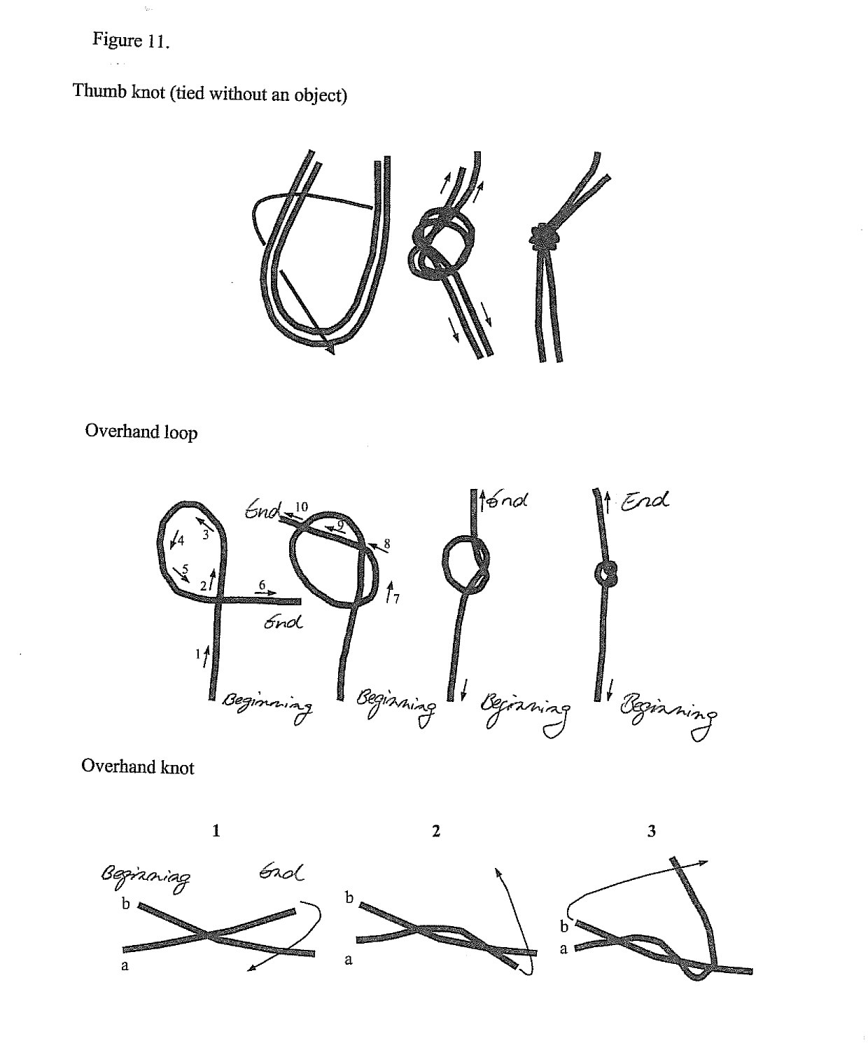

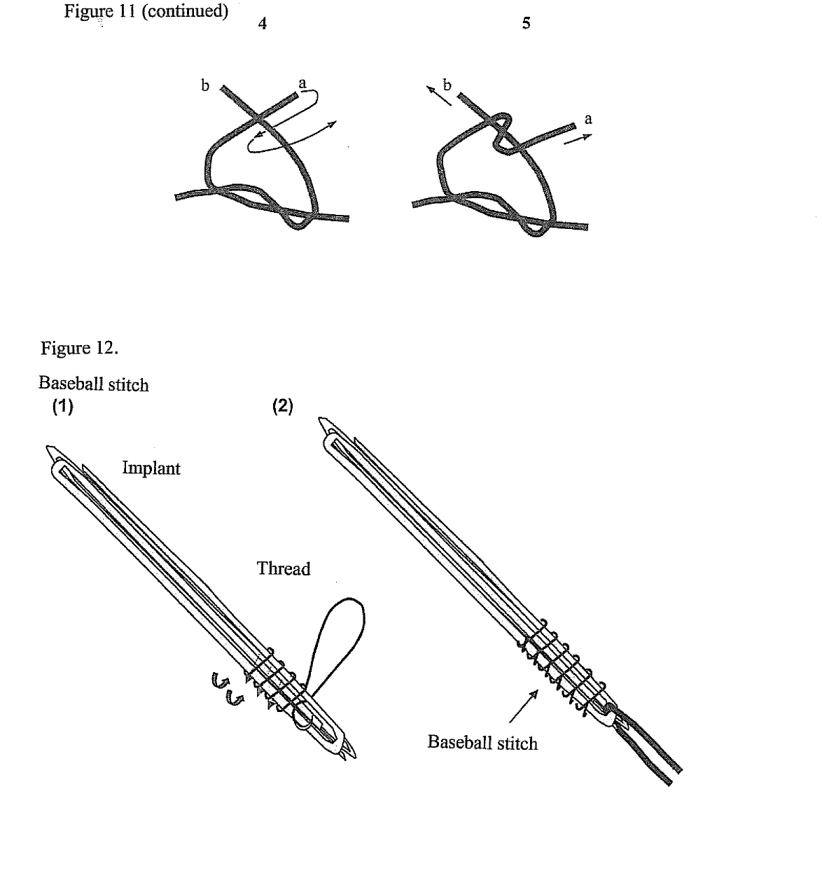

For producing collagen threads, the individual collagen fibers are knotted together with an overhand knot (FIG. 11).

In a first step 1, two collagen fibers are laid one over the other so as to yield an X. In the 2nd step the collagen fiber (a) on the bottom is laid over the upper collagen fiber (b), and collagen fiber (a) pulled through under collagen fiber (b) again. Then in the 3rd step the beginning of collagen fiber (b) is laid over the end of collagen fiber (a), and in step 4 the end of collagen fiber (b) laid first under and then over collagen fiber (b). Finally, in step 5 the collagen fibers (a) and (b) are carefully pulled in the opposite direction. Steps 3-5 can be repeated, so that a double overhand knot arises.

The collagen cords can be produced here from an individual “collagen thread” or from several parallel extending collagen threads.

The collagen threads can also be coiled in order to increase the tear strength and then be used directly as a collagen construct. The collagen cords can likewise be coiled in order to increase the tear strength and to be used directly as a collagen construct.

Additionally, the collagen cords can be turned/twisted, whereby the turned/twisted cords are partly additionally “flipped over” or “folded” to further increase the tear strength. Upon turning/twisting, individual and/or several parallel arranged collagen cords can be used. The tear strength of the thus produced construct can be increased further by several consecutive turning/twisting steps.

“Cruciate ligament type 2”: The collagen threads produced by knotting are interconnected directly by turning/twisting. Partly, the turned/twisted collagen threads are are subsequently additionally flipped over/folded. Again, individual or parallel arranged collagen threads can be used. The tear strength of the thus produced construct can be increased further by several consecutive turning/twisting steps. Upon twisting of the collagen threads, one end of a collagen thread is turned to the right and the other end turned to the left until a resistance arises. The twisted collagen threads can then be flipped over/folded/halved. In so doing, the two flipped over collagen thread strands turn around each other.

With both variants (“cruciate ligament type 1” and “cruciate ligament type 2”) there can currently be produced constructs with tear strengths per square millimeter of up to 60 N/mm². For a cruciate ligament construct with a diameter of 8 mm, i.e. a cross section of approx. 50 mm², there thus results a tear strength of up to 3000N.

Altogether there can thus be manufactured, in dependence on the diameter, cruciate ligament constructs with different tear strengths, e.g. a tear strength greater than 500 N, 500 to 1000 N, 1000 to 2000 N, 2000 to 3000 N, greater than 3000 N.

Different Tear Strengths Per Unit Area were Measured in the Collagen Fiber Constructs:
“Cruciate ligament construct 0”: (parallel single fibers)
16 N/mm², at a diameter of the construct of 8 mm this yields a tear strength of approx. 803N.
“Cruciate ligament construct 1” (collagen thread)
31 N/mm², at a diameter of the construct of 8 mm this yields a tear strength of 1557 N.
“Cruciate ligament construct 1” (collagen thread coiled)
28 N/mm², at a diameter of the construct of 8 mm this yields a tear strength of 1406 N.
“Cruciate ligament construct 1” (collagen cord)
60 N/mm², at a diameter of the construct of 8 mm this yields a tear strength of 3014 N.
“Cruciate ligament construct 2” (collagen thread twisted)
58 N/mm², at a diameter of the construct of 8 mm this yields a tear strength of 2913 N.
“Cruciate ligament construct 3” (collagen thread braided)
19 N/mm², at a diameter of the construct of 8 mm this yields a tear strength of 954 N.
“Cruciate ligament construct 4” (branched collagen fiber construct)
Partial strands depending on embodiment, see above.

### Example 6

### Seeding of Cruciate Ligament Construct with Cells after Grafting

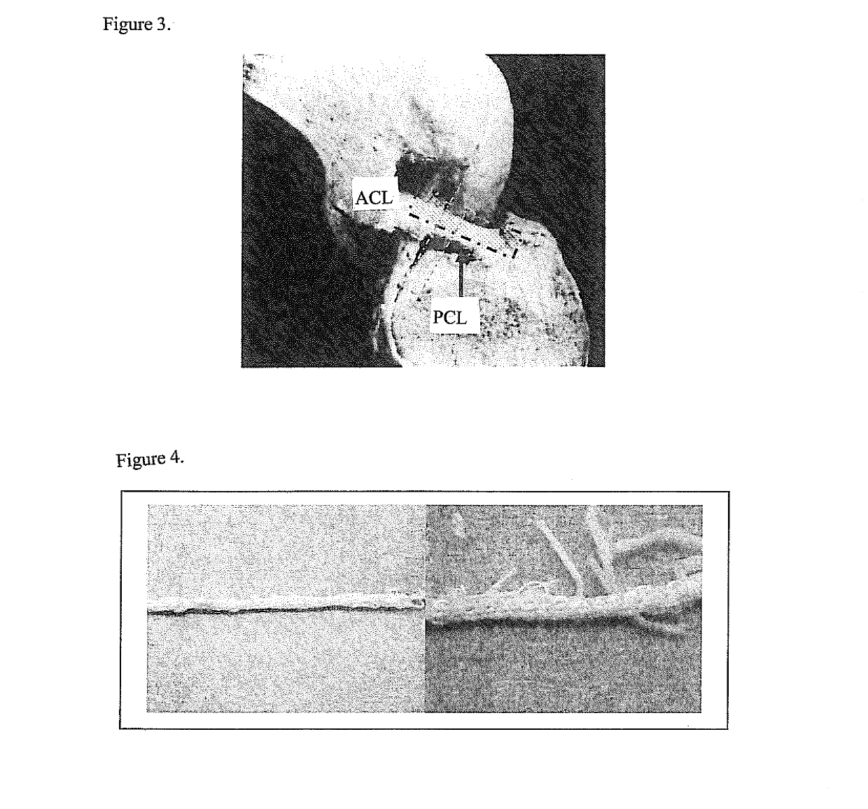

After implantation (FIG. 3) upon a rupture of the anterior cruciate ligament, the cruciate ligament construct is to be seeded by fibroblasts and epithelial cells. In so doing, different cells (primarily fibroblasts) attach to the implant, proliferate, migrate and form a ligamentary matrix. Furthermore, endothelial cells immigrate, which lead to vascularization.

### Example 7

### In Vivo Animal Study: Miniature Pig Implants

### 7.1 Manufacture of Collagen Fiber Constructs for Implantation

For the hereinafter described implantation, collagen fiber constructs were first manufactured, as described above, from collagen fibers which were connected into a collagen thread by knotting and subsequently knitted into a collagen cord (cruciate ligament type 1 which was additionally knitted into a collagen cord).

The cords were subsequently coiled several times and finally twisted (cruciate ligament type 2). The number of windings varies with the thickness of the collagen fibers used and is so chosen that the desired diameter of the collagen fiber construct is approximately obtained. The exact diameter is obtained by the final twisting. In so doing, two to twenty turns are used, depending on the requirements and the fibers used, since one must be careful when twisting not to compress the collagen fibers too strongly, since otherwise the contained water is pressed out and the fibers then become brittle. Accordingly, the pre-tension during twisting and the number of turns are manually so chosen that no, or only little, liquid passes out of the fibers.

The cruciate ligament implants were manufactured for use in a miniature-pig animal study and have the following dimensions: Length 3.9-4.1 cm, diameter of 3.0-3.2 mm when not fully loaded. The number of windings during manufacture depends on the thickness of the individual fibers, which in turn varies from rat tail to rat tail. Usually 13-17 windings were used for the desired diameter.

The tear strength of these cruciate ligament implants prior to implantation varies depending on the starting material used and lies in the range of 200 to 400 N. This results in a tear strength per unit area of 25 to 57 N/mm². For a cruciate ligament implant with a diameter of 8 mm, as is preferably to be manufactured for use in humans, this results in a tear strength of 1250 to 2844 N.

### 7.2 Implantation of the Cruciate Ligament Implants

Hereinafter there will be described the surgical technique with which the implants were implanted in the miniature pig. The cruciate ligament constructs are guided through bores in femur and tibia, fastened e.g. with an EndoButton/suture button or steel pin (cruciate ligament anchor). In the miniature pigs there were used so-called surgical loops (fabric tapes) to connect the collagen fiber constructs on both sides with the suture buttons (the cruciate ligament constructs were implanted in the miniature pig using suture buttons from Arthrex). In so doing, a fine adjustment of the total length of anchor/button-surgical loop-construct-surgical loop-anchor/button can be performed by the choice of the suitable length of the surgical loops. Moreover, one must make sure that there is respectively a sufficiently long piece of the collagen construct in the bone tunnels (femur and tibia) to make it possible for the constructs to engraft into the bones. Suitable lengths here are e.g. 1.5 to 4 cm on each side in humans and 1.0 to 2.5 cm in miniature pigs.

7.3 Results of the In Vivo Animal Study after about Six Months

### 7.3.1 Postoperative Phenotypic State of the Animals

Six weeks after implantation, the animals load the operated knee completely again and show only little or even no more limp or lameness. Directly after implantation, the animals relieve the operated leg. They load it only e.g. in the case of the flight reflex, but then completely right away, whereby a lameness or limp is to be recognized in individual animals. The outward appearance of all operated animals was normal. No infections or inflammations occurred in the region of the cruciate ligament constructs within the first six months after implantation. The animals all showed good wound healing, were active and ate as usual. A swelling of the operated knee was to be recognized, if at all, only in the first days after the operation.

After about six months after implantation, the animals were either examined biomechanically, as described below (see 7.3.3), or the implants were histologically processed and evaluated (see 7.3.2).

### 7.3.2 Histological Examination of the Implants

Histological examinations show an immigration of cells (e.g. fibroblasts) into the implant and the forming of blood vessels. Thus, the origination of a ligament-like tissue structure (ligamentization) can be inferred. Six months after implantation, the implants have developed into ligament-like constructs.

### 7.3.3 Biomechanical Examination

In the biomechanically examined animals, the tear strength of the collagen constructs lay in the range of the initial tear strength of the constructs (prior to implantation). The measured tear strength six months after implantation lay in the range of 222 to 385 N. The tear strength could thus be almost completely retained (>96%) or even increased (+11%). This results in a tear strength per unit area of 27.6 to 54.5 N/mm² based on the originally used diameter of 3.0 to 3.2 mm. For a cruciate ligament implant with a diameter of 8 mm, as is preferably to be manufactured for use in humans, there thus results a tear strength of 1388 to 2738 N.

### 7.3.4 Summary and Discussion of Results

The above-described animal study shows, inter alia, the following advantages:

All the animals of the above-described animal study have an intact “cruciate ligament replacement” after six months.

None of the animals showed an inflammatory reaction. All the constructs show a good to very good incorporation. By contrast, inflammatory reactions occurred in the constructs described in the prior art (Chvapil et al. (1993); see introduction) wherein the collagen fibers were treated with cross-linkers to increase the mechanical stability.

As described above, the tear strength of the collagen constructs lay in the range of the initial tear strength of the constructs prior to implantation or could even be increased. Thus, the herein-described constructs are characterized, in contrast to those described in the prior art (Chvapil et al. (1993); see introduction), by a constant tear strength and a very good incorporation potential. Thus, it is also possible with the herein-described constructs to realize a cruciate ligament replacement composed of pure collagen fibers, while this is ruled out in the publication by Chvapil et al. (1993). There, the use of synthetic fibers in addition to the collagen fibers is judged to be necessary for the mechanical stability. This is unnecessary in the herein-described constructs. Here, the collagen construct alone is sufficient.

The constructs described and used here are accepted well by the bodies of the laboratory animals and, moreover, a ligamentization could be observed (macroscopic observation), as described above. By contrast, the strongly purified and cross-linked constructs used by Chvapil et al. (1993) were hardly accepted by the body and yielded a poor incorporation.

### FIG. 1.

Length approx. 11-20 cm [bitte überprüfen, schwer leserlich]

### FIG. 2.

approx. 3 cm

### FIG. 3.

### ACL

### PCL

### FIG. 4.

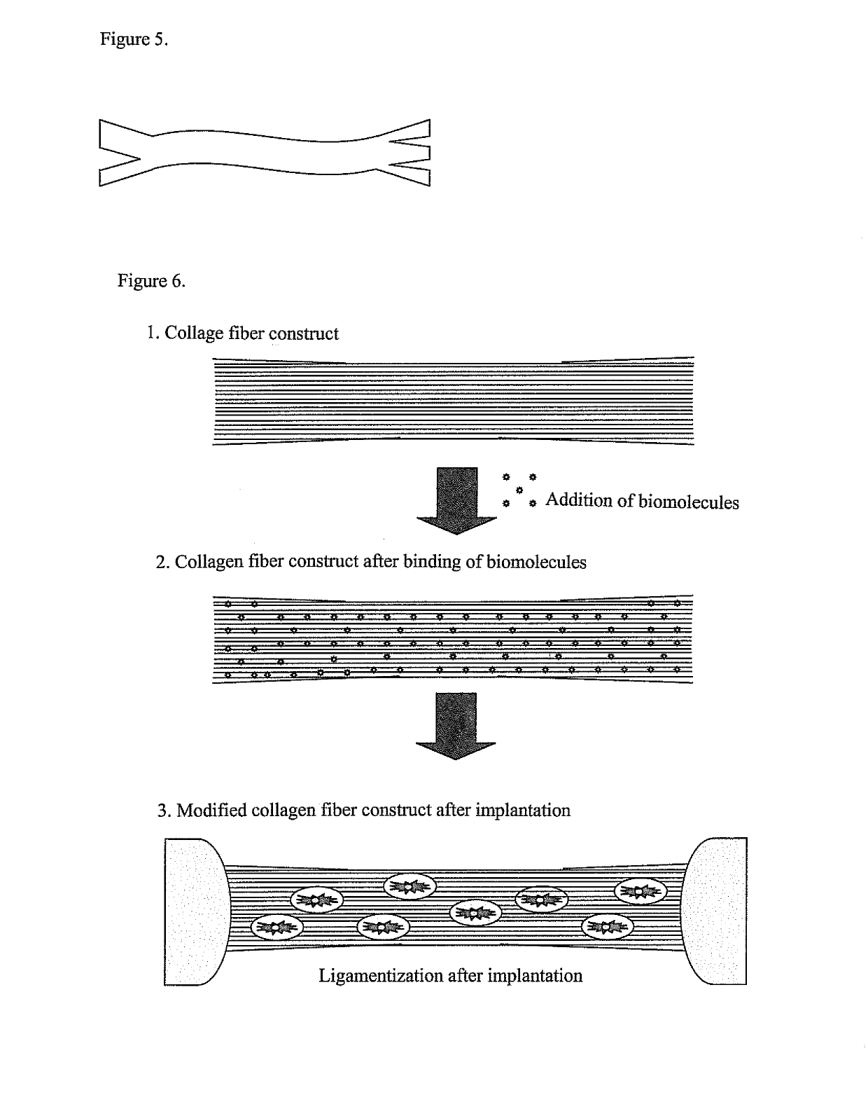

### FIG. 5.

### FIG. 6.

1. Collage fiber construct
Addition of biomolecules
2. Collagen fiber construct after binding of biomolecules
3. Modified collagen fiber construct after implantation
Ligamentization after implantation

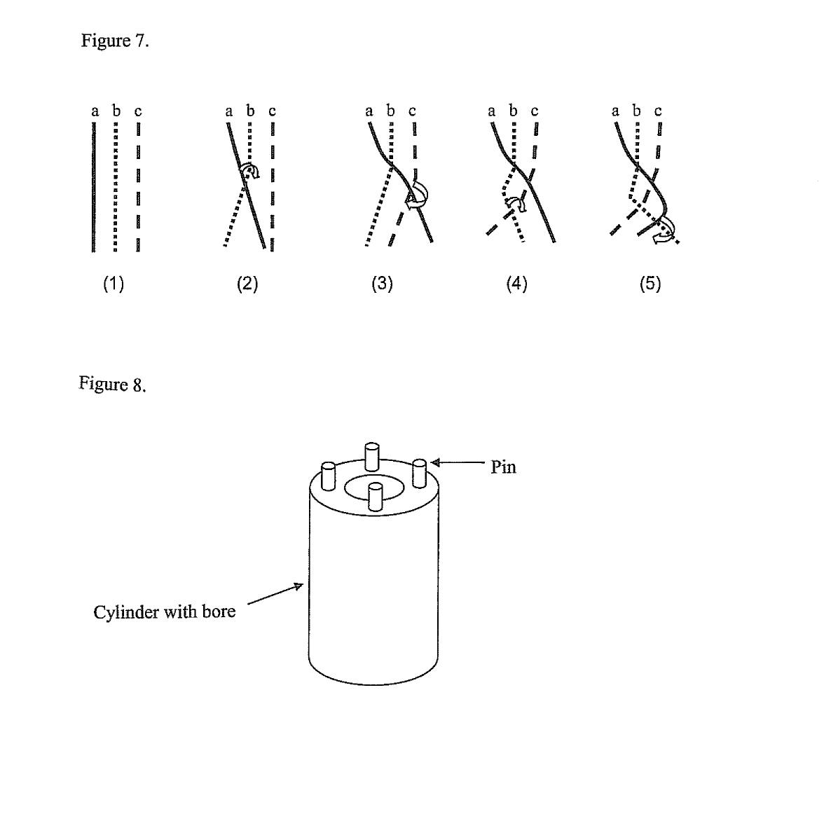

### FIG. 7.

### FIG. 8.

### Pin

Cylinder with bore

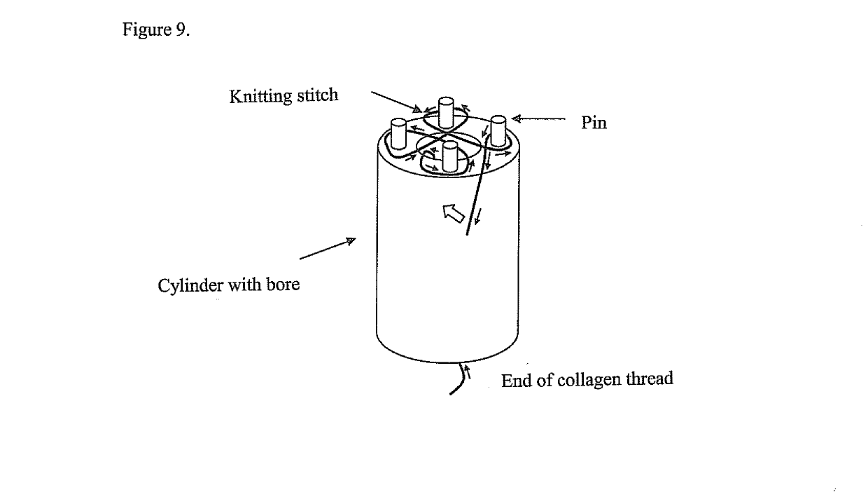

### FIG. 9.

Knitting stitch

### Pin

End of collagen thread
Cylinder with bore

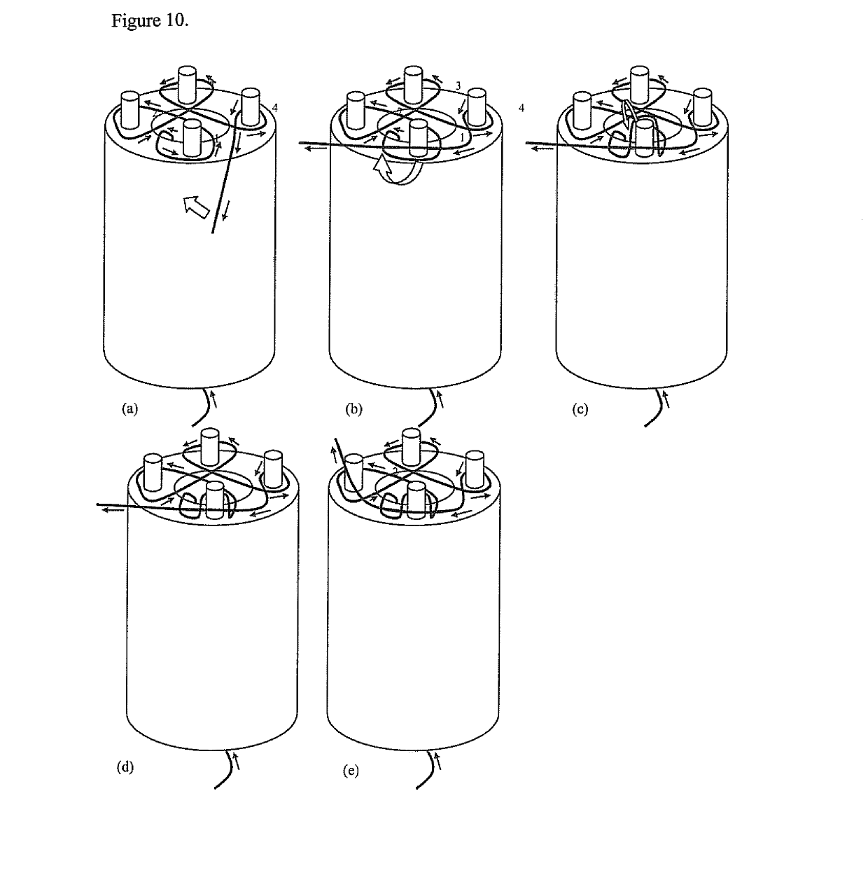

### FIG. 10.

### FIG. 11.

Thumb knot (tied without an object)
Overhand loop

### End

### Beginning

Overhand knot

### Beginning

### End

FIG. 11 (continued)

### FIG. 12.

Baseball stitch

### Implant

### Thread

Baseball stitch

1. A collagen fiber construct composed of single collagen fibers, which is sterilized with alcohol and/or via irradiation and not populated with cells, wherein the single collagen fibers are isolated from collagen-containing tissue from mammals.

2. The collagen fiber construct according to claim 1, wherein the collagen-containing tissue is isolated from rat tails.

3. The collagen fiber construct according to claim 1, wherein the collagen fiber construct is a cruciate ligament construct.

4. The collagen fiber construct according to claim 1, wherein the single collagen fiber is sterilized with 60% ethanol.

5. The collagen fiber construct according to claim 1, wherein several single collagen fibers are knotted into a collagen thread.

6. The collagen fiber construct according to claim 5, wherein one and/or several collagen threads are knitted into a collagen cord.

7. The collagen fiber construct according to claim 6, wherein the collagen cord is twisted and/or coiled.

8. The collagen fiber construct according to claim 7, wherein the twisted and/or coiled collagen cord is flipped over.

9. The collagen fiber construct according to claim 5, wherein one and/or several collagen cords are braided.

10. The collagen fiber construct according claim 5, wherein the collagen fiber construct is of branched structure.

11. The collagen fiber construct according to claim 1, wherein the collagen fiber construct is sterilized with gamma radiation.

12. The collagen fiber construct according to claim 1, wherein the cruciate ligament construct is an anterior and/or posterior cruciate ligament.

13. A method for manufacturing a collagen fiber construct composed of single collagen fibers, which is sterilized with alcohol and/or via irradiation and is not populated with cells, isolating the single collagen fibers from collagen-containing tissue from mammals.

14. The method according to claim 13, whereby the collagen-containing tissue is isolated from rat tails.

15. The method according to claim 13, wherein the collagen fiber construct is a cruciate ligament construct.

16. The method for manufacturing a collagen fiber construct according to claim 13, wherein the isolation and sterilization of the single collagen fibers and the manufacture of the collagen fiber constructs optionally comprises the steps of:
(a) isolating collagen-containing tissue;
(b) extracting individual and/or several single collagen fibers from the collagen-containing tissue;
(c) incubating the single collagen fibers in an isotonic/iso-osmolar solution;
(d) sterilizing the single collagen fibers in alcohol;
(e) optionally repeating the washing and sterilization steps according to points (c) and (d);
(f) sterilizing the collagen fiber construct in alcohol: and
(g) sterilizing the collagen fiber construct by irradiation.

17. The method according to claim 13, wherein the isolation of collagen-containing tissue comprises individual and/or several ones of the steps of:
(a) washing the rat tails with an isotonic/iso-osmolar solution;
(b) sterilizing the rat tails with alcohol;
(c) skinning the tails; and
(d) washing the skinned tails with a sterile isotonic/iso-osmolar solution.

18. The method according to claim 15, wherein individual and/or several single collagen fibers are knotted into a collagen thread.

19. The method according to claim 18, wherein individual and/or several collagen threads are knitted into a collagen cord.

20. The method according to claim 18, wherein the collagen cord or one or several collagen threads are twisted and/or coiled.

21. The method according to claim 20, wherein the twisted and/or coiled collagen cord or the twisted and/or coiled collagen threads are flipped over.

22. The method according to claim 18, wherein one and/or several collagen threads and/or collagen cords are braided.

23. The method according to claim 18, wherein the collagen fiber construct is of branched structure.

24. The method according to claim 13, wherein the collagen fiber construct is irradiated with gamma radiation.

25. The method according to claim 13, wherein the incubating and washing steps are preferably effected with isotonic/iso-osmolar washing solution.

26. The method according to claim 13, wherein the sterilization steps are preferably effected in 60% EtOH.

27. A collagen fiber construct manufactured by the method according to claim 13.

28. The collagen fiber construct according to claim 27, wherein the collagen fiber construct is a cruciate ligament construct.

29. A method for treating orthopedic diseases comprising implanting a collagen fiber construct of claim 1 in a patient having an orthopedic disease.

30. The collagen fiber construct according to claim 29, wherein the orthopedic disease is a cruciate ligament rupture, an Achilles tendon rupture, an injury and/or degeneration of the tendons of the rotator cuff (shoulder), an injury/rupture of the lateral collateral ligaments on the knee or on the ankle, or an injury/rupture or degeneration of the medial patellofemoral ligament (MPFL).

31. The collagen fiber construct according to claim 5, wherein the one or several collagen threads are twisted and/or coiled.

32. The collagen fiber construct according to claim 31, wherein the twisted and/or coiled collagen threads are flipped over.

33. The collagen fiber construct according to claim 5, wherein one and/or several collagen threads are braided.

34. The collagen fiber construct according to claim 5, wherein the collagen fiber construct is of branched structure.
